(19) United States
(12) Patent Application Publication
(10) Pub. No.: US 2023/0029678 A1
(43) Pub. Date: Feb. 2, 2023

(54) GENERATING CLUSTERED EVENT EPISODE BUNDLES FOR PRESENTATION AND ACTION

(71) Applicant: HEALTHPOINTE SOLUTIONS, INC., Austin, TX (US)

(72) Inventors: Nathan GNANASAMBANDAM, Irvine, CA (US); Mark Henry ANDERSON, Newport Coast, CA (US)

(73) Assignee: HEALTHPOINTE SOLUTIONS, INC., Austin, TX (US)

(21) Appl. No.: 17/790,920

(22) PCT Filed: Dec. 16, 2020
(86) PCT No.: PCT/US2020/065331

§ 371 Date: Jul. 5, 2022

Related U.S. Application Data
(60) Provisional application No. 62/957,673, filed on Jan. 6, 2020.

(51) Int. Cl. G16H 50/70; G16H 50/30; G16H 70/00; G16H 40/20

(52) U.S. Cl. CPC G16H 50/70; G16H 50/30; G16H 70/00; G16H 40/20

(57) ABSTRACT

In one embodiment, a method is disclosed for presenting a user interface including clustered event episode bundles pertaining to a medical condition. The method includes receiving, from a server, the clustered event episode bundles comprising event information clustered into an episode based on when events occurred and a taxonomy of ontological medical knowledge, presenting, via the user interface, a timeline that represents a period of time, and presenting, via the user interface, the clustered event episode bundles pertaining to each patient of a plurality of patients in a respective band relative to the timeline.

### CROSS-REFERENCE TO RELATED APPLICATIONS

This application claims the benefit of U.S. Provisional Application Ser. No. 62/957,673 filed Jan. 6, 2020 titled “Generating Clustered Event Episode Bundles for Presentation and Action,” which provisional application is incorporated by reference herein as if reproduced in full below.

### BACKGROUND

Population health management entails aggregating patient data across multiple health information technology resources, analyzing the data with reference to a single patient, and generating actionable items through which care providers can improve both clinical and financial outcomes. A population health management service seeks to improve the health outcomes of a group by improving clinical outcomes while lowering costs.

### SUMMARY

Representative embodiments set forth herein disclose various techniques for generating clustered event episode bundles for presentation and action.

In one embodiment, a method is disclosed for presenting a user interface including clustered event episode bundles pertaining to a medical condition. The method includes receiving, from a server, the clustered event episode bundles comprising event information clustered into an episode based on when events occurred and a taxonomy of ontological medical knowledge, presenting, via the user interface, a timeline that represents a period of time, and presenting, via the user interface, the clustered event episode bundles pertaining to each patient of a plurality of patients in a respective band relative to the timeline.

In some embodiments, a system may include a memory device storing instructions and a processing device communicatively coupled to the memory device. The processing device executes the instructions to perform any operation of the methods disclosed herein.

In some embodiments, a tangible, non-transitory computer-readable medium stores instructions that, when executed, cause a processing device to perform any operation of the methods disclosed herein.

### BRIEF DESCRIPTION OF THE DRAWINGS

For a detailed description of example embodiments, reference will now be made to the accompanying drawings in which:

FIG. 1 illustrates, in block diagram form, a system architecture 100 that can be configured to provide a population health management service, in accordance with various embodiments.

FIG. 2 shows additional details of a knowledge cloud, in accordance with various embodiments.

FIG. 3 shows an example subject matter ontology, in accordance with various embodiments.

FIG. 4 shows aspects of a conversation, in accordance with various embodiments.

FIG. 5 shows a cognitive map or “knowledge graph”, in accordance with various embodiments.

FIG. 6 shows an example block diagram performing mapping operations, in accordance with various embodiments.

FIG. 7 shows an example method for performing mapping operations to determine a knowledge fragment, in accordance with various embodiments.

FIG. 8 shows an example method for mapping a set of codes to a taxonomy of data to determine a utilization unit, in accordance with various embodiments.

FIG. 9 shows an example table used to cache data used or output by the method of FIG. 87, in accordance with various embodiments.

FIG. 10 shows an example table used to determine a confidence level of the determined utilization unit, in accordance with various embodiments.

FIG. 11 shows an example method for determining whether a utilization unit is correctly mapped, in accordance with various embodiments.

FIG. 12 shows an example user interface for population characteristic, in accordance with various embodiments.

FIG. 13 shows an example user interface for managing risk associated with a medical condition at a population level, in accordance with various embodiments.

FIG. 14 shows an example user interface presenting durational events for a patient, in accordance with various embodiments.

FIG. 15 shows an example user interface presenting a graphical element of event sequences for a patient over a certain time period, in accordance with various embodiments.

FIG. 16 shows an example method for using a population profile to perform an intervention, in accordance with various embodiments.

FIG. 17 shows an example method for performing the intervention based on a risk, in accordance with various embodiments.

FIG. 18 shows another example method for performing the intervention based on the risk, in accordance with various embodiments.

FIG. 19 shows an example method for updating an artificial-intelligence engine based on an effectiveness of an intervention, in accordance with various embodiments.

FIG. 20 shows an example user interface for selecting a shared medical condition state of interest to be monitored and/or acted upon at a population level, in accordance with various embodiments.

FIG. 21 shows an example method for selecting a shared medical condition state of interest to be monitored and/or acted upon at a population level, in accordance with various embodiments.

FIG. 22A shows an example user interface presenting a registry of people having a first shared medical condition state, recommended actions, and autonomously performed actions, in accordance with various embodiments.

FIG. 22B shows an example user interface presenting a registry of people having a second shared medical condition state, recommended actions, and autonomously performed actions, in accordance with various embodiments.

FIG. 23 shows an example method for segmenting a population into a registry and performing an action for the registry, in accordance with various embodiments.

FIG. 24 shows an example method for tracking encounters with a medical condition associated with a shared medical condition state and performing an action based on the encounters, in accordance with various embodiments.

FIG. 25 shows an example of determining an anchor event and creating an episode of clustered events around the anchor event, in accordance with various embodiments.

FIGS. 26A-B show example user interfaces presenting event episode bundles for various users on a timeline, and autonomously performed actions, in accordance with various embodiments.

FIG. 27 shows an example method for presenting clustered event episode bundles on a timeline, in accordance with various embodiments.

FIG. 28 shows an example method for receiving a selection of an event in a clustered event episode bundle and receiving an action instruction that is generated based on the event, in accordance with various embodiments.

FIG. 29 shows an example computer system.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

According to some embodiments, a cognitive intelligence platform integrates and consolidates data from various sources and entities and provides a population health management service. The cognitive intelligence platform has the ability to extract concepts, relationships, and draw conclusions from a given text posed in natural language (e.g., a passage, a sentence, a phrase, and a question) by performing conversational analysis which includes analyzing conversational context. For example, the cognitive intelligence platform has the ability to identify the relevance of a posed question to another question.

The benefits provided by the cognitive intelligence platform, in the context of healthcare, include freeing up physicians from focusing on day to day population health management. Thus a physician can focus on her core competency—which includes disease/risk diagnosis and prognosis and patient care. The cognitive intelligence platform provides the functionality of a health coach and includes a physician's directions in accordance with the medical community's recommended care protocols and also builds a systemic knowledge base for health management.

Accordingly, the cognitive intelligence platform implements an intuitive conversational cognitive agent that engages in a question and answering system that is human-like in tone and response. The described cognitive intelligence platform endeavors to compassionately solve goals, questions and challenges.

In addition, physicians often generate patient notes before, during, and/or after consultation with a patient. The patient notes may be included in an electronic medical record (EMR). When a patient returns for a subsequent visit, the physician may review numerous EMRs for the patient. Such a review process may be time consuming and inefficient. Insights may be hidden in the various EMRs and may result in the physician making an incorrect diagnosis. Further, it may involve the physician accessing numerous screens and performing multiple queries on a database to obtain the various EMRs. As a result, the computing device of the physician may waste computing resources by loading various screens and sending requests for EMR data to a server. The server that receives the requests may also waste computing resources by processing the numerous requests and transmitting numerous responses. In addition, network resources may be wasted by transmitting the requests and responses between the server and the client.

Accordingly, some embodiments of the present disclosure address the issues of reviewing the EMRs, by cognifying unstructured data. Unstructured data may include patient notes entered into one or more EMRs by a physician. The patient notes may explain symptoms described by the patient or detected by the physician, vital signs, recommended treatment, risks, prior health conditions, familial health history, and the like. The patient notes may include numerous strings of characters arranged into sentences. The sentences may be organized in one or more paragraphs. The sentences may be parsed and indicia may be identified. The indicia may include predicates, objectives, nouns, verbs, cardinals, ranges, keywords, phrases, numbers, concepts, or some combination thereof.

The indicia may be compared to one or more knowledge graphs that each represents health related information (e.g., a disease) and various characteristics of the health related information. The knowledge graph may also include how the various diseases are related to one another (e.g., bronchitis can lead to pneumonia). The knowledge graph may represent a model that includes individual elements (nodes) and predicates that describe properties and/or relationships between those individual elements. A logical structure (e.g., Nth order logic) may underlie the knowledge graph that uses the predicates to connect various individual elements. The knowledge graph and the logical structure may combine to form a language that recites facts, concepts, correlations, conclusions, propositions, and the like. The knowledge graph and the logical structure may be generated and updated continuously or on a periodic basis by an artificial intelligence engine with evidence-based guidelines, physician research, patient notes in EMRs, physician feedback, and so forth. The predicates and individual elements may be generated based on data that is input to the artificial intelligence engine. The data may include evidence-based guidelines that is obtained from a trusted source, such as a physician. The artificial intelligence engine may continuously learn based on input data (e.g., evidence-based guidelines, clinical trials, physician research, electronic medical records, etc.) and modify the individual elements and predicates.

For example, a physician may indicate that if a person has a blood sugar level of a certain amount and various other symptoms (e.g., unexplained weight loss, sweating, etc.), then that person has type 2 diabetes mellitus. Such a conclusion may be modeled in the knowledge graph and the logical structure as “Type 2 diabetes mellitus has symptoms of a blood sugar level of the certain amount and various other symptoms,” where “Type 2 diabetes mellitus,” “a blood sugar level of the certain amount,” and “various other symptoms” are individual elements in the knowledge graph, and “has symptoms of” is a predicate of the logical structure that relates the individual element “Type 2 diabetes mellitus” to the individual elements of “a blood sugar level of the certain amount” and “various other symptoms”.

The indicia extracted from the unstructured data may be correlated with one or more closely matching knowledge graphs by comparing similarities between the indicia and the individual elements. Tags related to possible health related information may be generated and associated with the indicia in the unstructured data. For example, the tags may specify “A leads to B” (where A is a health related information and B is another health related information), “B causes C” (where C is yet another health related information), “C has complications of D” (where D is yet another health related information), and so forth. These tags associated with the indicia may be correlated with the logical structure (e.g., predicates of the logical structure) based on structural similarity to generate cognified data. For example, if a person exhibits certain symptoms and has certain laboratory tests performed, then that person may have a certain medical condition (e.g., type 2 diabetes mellitus) that is identified in the knowledge graphs using the logical structures.

A pattern may be detected by identifying structural similarities between the tags and the logical structure in order to generate the cognified data. Cognification may refer to instilling intelligence into something. In the present disclosure, unstructured data may be cognified into cognified data by instilling intelligence into the unstructured data using the knowledge graph and the logical structure. The cognified data may include a summary of a health related condition of a patient, where the summary includes insights, conclusions, recommendations, identified gaps (e.g., in treatment, risk, quality of care, guidelines, etc.), and so forth.

The cognified data may be presented on a computing device of a physician. Instead of reading pages and pages of digital medical charts (EMRs) for a patient, the physician may read the cognified data that presents pointed summarized information that can be utilized to more efficiently and effectively treat the patient. As a result, computing resources may be saved by preventing numerous searches for EMRs and preventing accessing numerous screens displaying the EMRs. In some embodiments, the physician may submit feedback pertaining to whether or not the cognified data is accurate for the patient. The feedback may be used to update the artificial intelligence engine that uses the knowledge graph and logical structure to generate the cognified data.

In some embodiments, the cognified data may be used to diagnose a medical condition of the patient. For example, the medical condition may be diagnosed if a threshold criteria is satisfied. The threshold criteria may include matching a certain number of predicates and tags for a particular medical condition represented by a particular knowledge graph. The computing device of the physician and/or the patient may present the diagnosis and a degree of certainty based on the threshold criteria. In some embodiments, the physician may submit feedback pertaining to whether or not the diagnosis is accurate for the patient. The feedback may be used to update the artificial intelligence engine that uses the knowledge graph and logical structure to generate the diagnosis using the cognified data.

Further, patients may be inundated with information about a particular medical condition with which they are diagnosed and/or inquiring about. The information may not be relevant to a particular stage of the medical condition. The amount of information may waste memory resources of the computing device of the patient. Also, the user may have a bad experience using the computing device due to the overwhelming amount of information.

In some embodiments, user experience of using a computing device may be enhanced by running an application that performs various techniques described herein. The user may be interacting with the cognitive agent and the cognitive agent may be steering the conversation as described herein. In some embodiments, the cognitive agent may provide recommendations based on the text entered by the user, and/or patient notes in EMRs, which may be transformed into cognified data. The application may present health related information, such as the cognified data, pertaining to the medical condition to the computing device of the patient and/or the physician.

Instead of overwhelming the patient with massive amounts of information about the medical condition, the distribution of information may be regulated to the computing device of the patient and/or the physician. For example, if the patient is diagnosed as having type 2 diabetes mellitus, a controlled traversing of the knowledge graph associated with type 2 diabetes mellitus may be performed to provide information to the patient. The traversal may begin at a root node of the knowledge graph and first health related information may be provided to the computing device of the patient at a first time. The first health related information may pertain to a name of the medical condition, a definition of the possible medical condition, or some combination thereof. At a second time, health related information associated with a second node of the knowledge graph may be provided to the computing device of the patient. The second health related information may pertain to how the medical condition affects people, signs and symptoms of the medical condition, a way to treat the medical condition, complications of the medical condition, a progression of the medical condition, or some combination thereof. The health related information associated with the remaining nodes in the knowledge graph may be distributed to the computing device of the patient at different respective times. In some embodiments, the health related information to be provided and/or the times at which the health related information is provided may be selected based on relevancy to a stage of the medical condition of the patient.

In other scenarios, users (also referred to as patients herein) may use various computing devices (e.g., smartphone, tablet, laptop, etc.) to schedule an appointment with a person (also referred to as care providers herein) having a particular specialty to perform a service. For example, a patient may schedule appointments with care providers to provide one or more services to the patient. A patient may call an office where the care provider having a specialty works and speak to a person who finds an available appointment to book for the care provider and the patient. To book an appointment with another care provider having a different specialty, the patient may call the office of the other care provider having the different specialty to book an available appointment. Further, to book an appointment with a care provider for a dependent (e.g., child), the parent/guardian may contact yet another office where a care provider having yet another specialty (e.g., pediatrician) works to book an appointment. In some instances, the patient may access multiple different websites associated with the care providers to attempt to schedule an appointment. This is inconvenient for the patient and wastes resources by making multiple phone calls or accessing multiple different websites. Switching between websites to find contact information for people having different specialties may cause undesirable network, computing, and/or memory usage to occur. Additionally, typical software applications do not include functionality for scheduling appointments for an entire family (e.g., primary, spouse, dependents (children, senior citizens)) covered by an insurance plan, and/or functionality for scheduling multiple appointments for the same patient and/or different patients.

When the patient arrives for the scheduled appointments, the patient typically has to fill out paper check-in documents at each office. Even when the information requested by the check-in documents is redundant, such as medical history information, medication information, etc., various offices still request the same information. Part of the issue is a lack of interoperability of electronic medical records systems. Also, when a computing device is used to complete the check-in documents, the check-in documents are not shared with other systems associated with other specialties, and the user may have to reenter their information using a computing device of another system associated with the other specialties. As such, computing resources of the computing devices may be wasted by running an application to enable entry of information into the check-in documents, instead of just sharing the already completed check-in documents with requesting systems.

Once check-in is complete, the patient may be presented with paper reading materials in a waiting room. The reading materials may include information (e.g., symptoms, causes, treatments, etc.) pertaining to various different medical conditions. It can oftentimes be overwhelming to a patient to be presented with too much information, especially when the information does not pertain to the condition or conditions for which the patient is seeking treatment. Further, even if the patient knows what he or she is looking for, searching for the paper reading material is inefficient. To that end, even if the user finds reading material that discusses a desired topic, there typically is not a guarantee the reading material was authored/reviewed by a person having proper credentials (e.g., a medical doctor). Educating the patient with pertinent curated content that is tailored for the patient is desired.

Accordingly, some embodiments of the present disclosure address the above-identified issues, among other things. For example, an autonomous multipurpose application may execute in a cognitive intelligence platform. In some embodiments, the autonomous multipurpose application may be implemented as one or more application programming interfaces (API) executing via one or more computing devices (e.g., servers), as described in more detail below. The term “autonomous” used in conjunction with the “multipurpose application” may refer to the multipurpose application executing a set of operations on behalf of a person or another application with some degree of independence or autonomy in an intelligent manner using knowledge or representation of a user's goals or desires. The terms “autonomous multipurpose application” and “cognitive agent” may be used interchangeably herein.

In some embodiments, the autonomous multipurpose application may present different user interfaces based on a role associated with a person that logs into the autonomous multipurpose application. The various roles may include a medical personnel (e.g., medical doctor, physician, nurse, dentist, optometrist, psychiatrist, behavioral specialist, physician assistant, and the like), an administrator, a patient/user, and so forth. The user interface presented on a computing device when a person having the medical personnel role is logged in may be referred to as “clinic viewer” herein. The user interface presented on a computing device when a person having the administrator role is logged in may be referred to as “administrator viewer” herein. The user interface presented on a computing device when a person having the patient/user role may be referred to as “patient viewer” herein.

The autonomous multipurpose application may perform numerous actions or operations pertaining to scheduling appointments for patients, checking-in patients for scheduled appointments, educating the patients about medical conditions, and/or searching for content based on search queries, among other things. For scheduling purposes, the autonomous multipurpose application may be communicatively coupled with computing devices of care providers (e.g., medical personnel) and/or electronic medical record (EMR) systems used by the care providers (e.g., medical personnel). These computing devices and/or electronic medical record systems may execute patient management systems or scheduling management systems that maintain schedules of appointments for the care providers. For example, a schedule for a care provider may show which appointments are scheduled or booked and which appointments are available by date and time.

The autonomous multipurpose application may obtain the schedules for people having a desired specialty within a certain geographic location (e.g., within a radius of a geolocation of a computing device of the user, within a radius of an entered address, etc.). A user may elect to enable electronic scheduling. If an available appointment is found within the certain geographic region, and the user is available at the same date and time as the available appointment, the autonomous multipurpose application may electronically schedule the available appointment as a booked appointment. If the user has not enabled electronic scheduling, the autonomous multipurpose application may recommend one or more available appointments to the computing device of the user for presentation.

The autonomous multipurpose application may enable a user to schedule numerous appointments for himself or herself with people having different specialties via a single user interface. For example, the specialties may include a medical doctor (physician), a dentist, an optometrist, a physician's assistant, a chiropractor, a behavioral specialist, a lab technician, a masseuse, a barber, an orthodontist, a dermatologist, and the like. Also, the autonomous multipurpose application may enable the user to schedule appointments for dependents (e.g., children, spouse, senior citizen, etc.) of an insurance plan.

In some embodiments, the autonomous multipurpose application may provide service cost transparency. For example, the autonomous multipurpose application may use the insurance plan information extracted from an insurance card and/or provided by a user to determine what a service may cost. The autonomous multipurpose application may determine a co-pay cost based on the deductible of the insurance plan. The autonomous multipurpose application may determine a self-pay cost without considering the insurance plan. The co-pay cost and the self-pay cost may be presented on the computing device of the user, administrator, or person having a specialty. In some embodiments, if electronic scheduling is enabled, the autonomous multipurpose application may electronically select the cost that is the lowest.

Further, the autonomous multipurpose application may function as a centralized manager and repository for documents pertaining to the user and the dependents of the user. For example, when a user checks-in using a computing device (e.g., kiosk) executing the autonomous multipurpose application at a clinic, check-in documents pertaining to the user stored in a database may be checked to determine whether the check-in documents are complete. The check-in documents may refer to consent forms, medical history documents, health information release authorization forms, new patient sheets, massage client intake forms, mental health intake forms, consent treatment for minor child forms, doctor referral forms, adult health history forms, school physical forms, insurance verification sheets, medical reports, therapy intake forms, initial exam reports, pain assessment sheets, and the like. In some embodiments, the autonomous multipurpose application may communicate with external systems, such as EMR systems, to request the documents for the user from those systems. For example, if the user checked-in for another appointment with a different physician, the user may have already completed the various check-in documents and the autonomous multipurpose application may retrieve those completed check-in documents and store them for future reference. The autonomous multipurpose application may transmit the completed check-in documents to the EMR system associated with the person with which the user has an appointment.

If the check-in documents are partially complete, the autonomous multipurpose application may cause the portions of information that are missing to be presented for completion. If the check-in documents are incomplete, the autonomous multipurpose application may cause the check-in documents to be presented on a computing device for completion by the user, an administrator, a person having a specialty, or the like.

The autonomous multipurpose application may also manage and store other information for the users. For example, the user may capture an image of their driver's license, insurance card, and the like, and transmit the image to the autonomous multipurpose application. The autonomous multipurpose application may analyze the image (e.g., using machine learning and/or optical character recognition) to extract information from the image. For example, the autonomous multipurpose application may extract a picture of the user from a driver's license, a name of the user, a birthdate of the user, an address of the user, an identification number, an insurance plan number, a type of insurance, an expiration date of the user's driver's license, an expiration date of the user's insurance plan, and the like. The autonomous multipurpose application may electronically fill information in corresponding documents based on the extracted information. Further, the autonomous multipurpose application may perform logic based on the extracted information. For example, if the user's insurance is about to expire, the autonomous multipurpose application may transmit a message (e.g., email, text message, phone call, onscreen notification, etc.) to the user to renew their insurance. Similar types of information may be managed and stored for each person in a family. The information may be disbursed to a requesting client, such as an EMR system used by an entity at which the users make appointments.

The autonomous multipurpose application may communicate with a knowledge cloud that includes knowledge graphs that each pertain to a respective medical condition. For example, each knowledge graph may include individual elements (e.g., health artifacts) and predicates that describe relationships between the individual elements in a logical structure. Each knowledge graph may include nodes representing the individual elements and branches representing the predicates that connect the nodes. Each knowledge graph may begin at a root node that includes a type or name of the medical condition, for example. One knowledge graph may include a root node representing “Diabetes”. A predicate may represent “is caused by” branch that connects to another node “high blood sugar”. The logical structure may be formulated as “Diabetes is caused by high blood sugar”.

When a user successfully checks-in for a scheduled appointment, the autonomous multipurpose application may access the knowledge cloud to obtain curated content pertaining to one or more conditions of the user. For example, the user may specify the condition for which the user is seeking treatment, and educational curated content about that condition may be recommended and/or provided to the computing device of the user. The autonomous multipurpose application may also recommend other curated content to the user for the medical conditions of the user that are known by the autonomous multipurpose application. Each time a user has an appointment, the autonomous multipurpose application may update information pertaining to the user to keep knowledge about the user up to date.

In addition, when the user is checked-in, a wait time estimator model may be used by the autonomous multipurpose application to provide an estimated wait time. For example, the wait time estimator may be a machine learning model that is trained using data representing an average amount of time it takes a person having a specialty to perform a service. The training data may be specific for each different person and the amount of time it takes that person to perform the service. The wait time estimator may use training data pertaining to each patient. For example, if John Smith is at an appointment in the doctor's office immediately before Jane Doe, the average time that John Smith stays in the office may be used to estimate the wait time for Jane Doe. The wait times from different offices and/or clinics may be aggregated for each specialty in that office and/or for each person having the specialties to perform the service associated with the specialties.

Various timestamps associated with interactions between the user and the person having the specialty may be obtained from a system (e.g., EMR) used by the person having the specialty. For example, a timestamp of when the user checked-in for a scheduled appointment may be obtained, a timestamp of how long it took for the user to be called back to the doctor's office may be obtained, a timestamp of how long the user waited in the doctor's office prior to the doctor entering, a timestamp of any patient notes made by the doctor, a timestamp of any patient notes made by a nurse, a timestamp of when the doctor leaves after performing a service, a timestamp of when the user pays, or some combination thereof. The timestamps may be used to estimate wait times for users that have appointments scheduled with that doctor.

The autonomous multipurpose application may provide natural language searching for content. For example, the user may search “information about Diabetes” and the autonomous multipurpose application may return curated content pertaining to Diabetes to the computing device of the user.

The disclosed autonomous multipurpose application may provide an enhanced experience for users by improving scheduling, check-in, wait time estimation, cost transparency, and/or content distribution, among other things. The autonomous multipurpose application may use artificial intelligence to make decisions and perform actions.

In addition, the cognitive intelligence platform may use a knowledge graph pertaining to a condition of a user and a data structure (e.g., a patient graph) corresponding to the condition and the user to electronically generate a care plan for the condition of the user. The patient graph may include elements (e.g., health artifacts) and branches representing relationships between the elements. The elements may be represented as nodes in the patient graph. The elements may represent interactions and/or actions the user has had and/or performed pertaining to the condition. For example, if the condition is diabetes and the user has already performed a blood glucose test, then the user may have a patient graph corresponding to diabetes that includes an element for the blood glucose test. The element may include one or more associated information, such as a timestamp of when the blood glucose test was taken, if it was performed at-home or at a care provider, a result of the blood glucose test, and so forth.

The autonomous multipurpose application may cause the patient viewer to be presented on the computing device of the user, and the patient viewer may present the various conditions of the user. Further, the patient viewer may ask the user to specify a number of areas of the condition the user would like to manage, and to select which areas of the condition the user would like to manage.

The patient graph for the condition of the user may be compared (e.g., projected on) to the knowledge graph for the condition of the user to generate a care plan. The cognitive intelligence platform may generate the care plan based on the areas of the condition the user specified to manage, based on areas of the condition on which the user has not taken action and/or interacted with in view of the knowledge graph and patient graph, based on a detected emotion of the user, based on a detected tone of the user, based on a medical outcome selected by a medical personnel, or some combination thereof. For example, the cognitive intelligence platform may determine that the user currently is prescribed medication A for diabetes based on the user's patient graph for diabetes, but medication A is ineffective for the user. The cognitive intelligence platform may compare the patient graph to the knowledge graph pertaining to diabetes to determine that medication B can be prescribed to treat diabetes for the user. The care plan may include an action instruction that instructs the medical personnel to prescribe medication B and/or discuss information pertaining to medication A and/or medication B. The care plan may be transmitted to the user device for presentation in the patient viewer, the clinic viewer, and/or the administrator viewer.

The patient graph for each condition may also include an engagement profile that may be used to determine a compliance of the user with the care plan. The engagement profile may store information at a meta data level that corresponds to the actions and/or interactions the user performs pertaining to the care plan for the condition. In some embodiments, activity of the user on the computing device may be tracked; medical records may be obtained from EMR systems, claims systems, clinical systems, and the like; and so forth. For example, if the care plan recommends the user read a certain article pertaining to diabetes, and the user selects the article, the engagement profile may store information related to the user selecting the article, how long the user read the article, if the user finished the article, and so forth. Further, if the medical records indicate the user had a blood glucose test performed, the engagement profile may store information pertaining to the blood glucose test being performed.

The patient graph for the diabetes of the user may be updated based on the information stored in the engagement profile. For example, if information in the engagement profile indicates the user completes performance of a blood glucose test, an element pertaining to the blood glucose test may be added to a section of the patient graph of the user corresponding to diabetes. In some embodiments, certain conditions may specify the same elements as each other. For example, two conditions may include knowledge graphs that both include elements for testing for the condition using a blood glucose test. If the patient performs the blood glucose test for one of the conditions, the patient graphs for both conditions may be updated to include the information for the blood glucose test at the appropriate elements. As a result, if a knowledge graph for one condition includes an element for a test, and the user has already performed the test for another condition, as represented in the patient graph for the other condition, the cognitive intelligence platform may not include an action instruction to perform the test in the care plan for the user for the one condition. In this way, the care plans may be not include redundant data and/or action instructions.

In some embodiments, the patient graph may represent a checklist of items (e.g., elements, actions, interactions, content, etc.) pertaining to the condition that the user performed. The knowledge graph may represent a superset of items pertaining to the condition, and if the user complies with the superset of items (e.g., completes a care plan for a condition), the user may be managing the condition in a desired manner (e.g., the user is taking medications on a specified basis, the values of certain tests for the user are within a desired range, the user has been informed by the recommended content, etc.). The compliance with the care plan may be determined based on the engagement profile and/or the patient graph.

In some embodiments, the patient graph for a condition may be compared (e.g., projected on) to the knowledge graph for the condition, and if the patient graph includes each element of the knowledge graph, then a determination may be made that the user is managing the condition in a desired manner. In some embodiments, a notification may be presented on the patient viewer, the clinic viewer, and/or the administrator viewer indicating the same. If some of the elements of the knowledge graph are missing in the patient graph, the cognitive intelligence platform may provide a care plan including action instructions pertaining to those missing elements. Based on the engagement profile, if certain elements are partially completed, performed, and/or interacted with, the cognitive intelligence platform may provide a care plan including action instructions pertaining to those partially completed, performed, and/or interact with elements.

In some embodiments, an emotion of the user, a tone of the user, and/or a medical outcome desired by a medical personnel may be used to modify the care plan presented to the user. For example, data (e.g., video, image, text, etc.) may be received by the cognitive intelligence platform from a computing device of the user while the user is interacting with the patient viewer and/or interacting with the computing device of the user. The cognitive intelligence platform may perform certain emotion detecting and/or tone detecting techniques using the data. For example, facial recognition techniques may be performed to determine an emotion the user is experiencing. Such a determination may be made in response to the care plan presented to the user, content presented to the user, responses provided by the cognitive intelligence platform, or the like. Further, a tone and/or emotion of the user may be determined using text input by the user while interacting with the patient viewer and/or interacting with the computing device of the user. In addition, the cognitive intelligence platform may receive a desired medical outcome input by a medical personnel using the clinic viewer.

The cognitive intelligence platform may modify the care plan based on the detected emotion, detected tone, and/or the desired medical outcome. The modified care plan may be presented in the patient viewer, the clinic viewer, and/or the administrator viewer.

In some embodiments, a clinic viewer may be generated and/or presented by the cognitive intelligence platform on a computing device of a care provider (e.g., medical personnel). The clinic viewer may display a reason that a patient scheduled an appointment. The clinic viewer may display a condition with which a patient has been diagnosed. The clinic viewer may display a care plan for the patient. The clinic viewer may display a recommendation to prescribe a certain dosage of a certain medication to the patient based on the patient's condition and vital statistics. The clinic viewer may display a recommended action for medical personnel to take when the patient visits. The clinic viewer may display information about current medication that the patient is taking. The clinic viewer may display a notification that medication that a patient is currently taking is incompatible with another medication that relates to the condition of the patient. The clinic viewer may display a recommendation that the medical personnel perform a service for the patient. The clinic viewer may display a quality of care recommendation and an evidence trail that explains why the quality of care recommendation was made. The clinic viewer may display curated content, such as medical journal articles, related to the patient's condition. The clinic viewer may display a user interface in which the medical personnel can update information about the clinic. The clinic viewer may display current and prior information about the patient. The clinic viewer may display a knowledge graph about the patient's condition and a patient graph specific for the patient having the condition. The clinic viewer may allow medical personnel to input medical information about the patient. The clinic viewer may be configured to allow medical personnel to schedule a future appointment with the patient. The clinic viewer may be configured to allow medical personnel to send a prescription for the patient to a pharmacy. The clinic viewer may be configured to allow medical personnel to schedule an appointment for the patient at another medical provider.

Medical personnel may be inundated with massive amounts of information about medical conditions and patient related information, which may delay the diagnosis decision making process or the process in determining what advice to provide to the patient. Doctors desire to analyze large quantities of data and need a mechanism to take clinical data that relates to a patient (especially long durational items) and apply a knowledge graph of ontological medical information pertaining to medical conditions to come up with a variety of action-oriented informatics. The present disclosure provides such a mechanism that aids in making sense of data. The present disclosure presents embodiments that may be akin to a self-driving car for a doctor that provides autonomous actions for registries of people having shared medical condition states and presenting clustered event episode bundles in a user friendly interactive dashboard.

The described methods and systems are described as occurring in the healthcare space, though other areas are also contemplated, such as finance, career, etc.

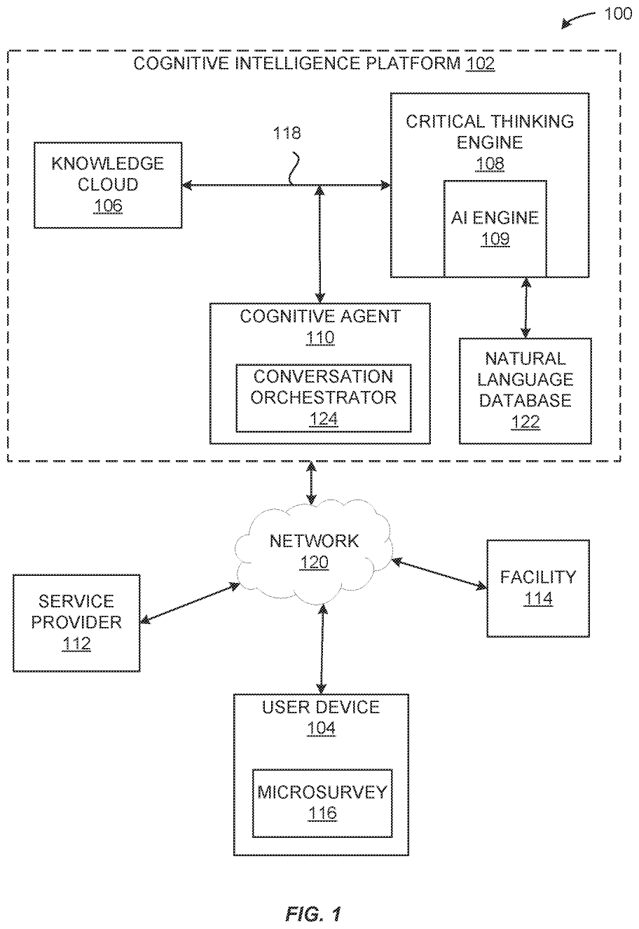

FIG. 1 shows a system architecture 100 that can be configured to provide a population health management service, in accordance with various embodiments. Specifically, FIG. 1 illustrates a high-level overview of an overall architecture that includes a cognitive intelligence platform 102 communicably coupled to a user device 104. The cognitive intelligence platform 102 includes several computing devices, where each computing device, respectively, includes at least one processor, at least one memory, and at least one storage (e.g., a hard drive, a solid-state storage device, a mass storage device, and a remote storage device). The individual computing devices can represent any form of a computing device such as a desktop computing device, a rack-mounted computing device, and a server device. The foregoing example computing devices are not meant to be limiting. On the contrary, individual computing devices implementing the cognitive intelligence platform 102 can represent any form of computing device without departing from the scope of this disclosure.

The several computing devices work in conjunction to implement components of the cognitive intelligence platform 102 including: a knowledge cloud 106; a critical thinking engine 108; a natural language database 122; and a cognitive agent 110. The cognitive intelligence platform 102 is not limited to implementing only these components, or in the manner described in FIG. 1. That is, other system architectures can be implemented, with different or additional components, without departing from the scope of this disclosure. The example system architecture 100 illustrates one way to implement the methods and techniques described herein.

The knowledge cloud 106 represents a set of instructions executing within the cognitive intelligence platform 102 that implement a database configured to receive inputs from several sources and entities. For example, some of the sources and entities include a service provider 112, a facility 114, and a microsurvey 116—each described further below.

The critical thinking engine 108 represents a set of instructions executing within the cognitive intelligence platform 102 that execute tasks using artificial intelligence, such as recognizing and interpreting natural language (e.g., performing conversational analysis), and making decisions in a linear manner (e.g., in a manner similar to how the human left brain processes information). Specifically, an ability of the cognitive intelligence platform 102 to understand natural language is powered by the critical thinking engine 108. In various embodiments, the critical thinking engine 108 includes a natural language database 122. The natural language database 122 includes data curated over at least thirty years by linguists and computer data scientists, including data related to speech patterns, speech equivalents, and algorithms directed to parsing sentence structure.

Furthermore, the critical thinking engine 108 is configured to deduce causal relationships given a particular set of data, where the critical thinking engine 108 is capable of taking the individual data in the particular set, arranging the individual data in a logical order, deducing a causal relationship between each of the data, and drawing a conclusion. The ability to deduce a causal relationship and draw a conclusion (referred to herein as a “causal” analysis) is in direct contrast to other implementations of artificial intelligence that mimic the human left brain processes. For example, the other implementations can take the individual data and analyze the data to deduce properties of the data or statistics associated with the data (referred to herein as an “analytical” analysis). However, these other implementations are unable to perform a causal analysis—that is, deduce a causal relationship and draw a conclusion from the particular set of data. As described further below—the critical thinking engine 108 is capable of performing both types of analysis: causal and analytical.

In some embodiments, the critical thinking engine 108 includes an artificial intelligence engine 109 (“AI Engine” in FIG. 1) that uses one or more machine learning models. The one or more machine learning models may be generated by a training engine and may be implemented in computer instructions that are executable by one or more processing device of the training engine, the artificial intelligence engine 109, another server, and/or the user device 104. To generate the one or more machine learning models, the training engine may train, test, and validate the one or more machine learning models. The training engine may be a rackmount server, a router computer, a personal computer, a portable digital assistant, a smartphone, a laptop computer, a tablet computer, a camera, a video camera, a netbook, a desktop computer, a media center, or any combination of the above. The one or more machine learning models may refer to model artifacts that are created by the training engine using training data that includes training inputs and corresponding target outputs. The training engine may find patterns in the training data that map the training input to the target output, and generate the machine learning models that capture these patterns.

The one or more machine learning models may be trained to generate one or more knowledge graphs each pertaining to a particular medical condition. The knowledge graphs may include individual elements (nodes) that are linked via predicates of a logical structure. The logical structure may use any suitable order of logic (e.g., higher order logic and/or Nth order logic). Higher order logic may be used to admit quantification over sets that are nested arbitrarily deep. Higher order logic may refer to a union of first-, second-, third, . . . , Nth order logic. Clinical-based evidence, clinical trials, physician research, and the like that includes various information (e.g., knowledge) pertaining to different medical conditions may be input as training data to the one or more machine learning models. The information may pertain to facts, properties, attributes, concepts, conclusions, risks, correlations, complications, etc. of the medical conditions. Keywords, phrases, sentences, cardinals, numbers, values, objectives, nouns, verbs, concepts, and so forth may be specified (e.g., labeled) in the information such that the machine learning models learn which ones are associated with the medical conditions. The information may specify predicates that correlates the information in a logical structure such that the machine learning models learn the logical structure associated with the medical conditions.

In some embodiments, the one or more machine learning models may be trained to transform input unstructured data (e.g., patient notes) into cognified data using the knowledge graph and the logical structure. The machine learning models may identify indicia in the unstructured data and compare the indicia to the knowledge graphs to generate possible health related information (e.g., tags) pertaining to the patient. The possible health related information may be associated with the indicia in the unstructured data. The one or more machine learning models may also identify, using the logical structure, a structural similarity of the possible health related information and a known predicate in the logical structure. The structural similarity between the possible health related information and the known predicate may enable identifying a pattern (e.g., treatment patterns, education and content patterns, order patterns, referral patterns, quality of care patterns, risk adjustment patterns, etc.). The one or more machine learning models may generate the cognified data based on the structural similarity and/or the pattern identified. Accordingly, the machine learning models may use a combination of knowledge graphs, logical structures, structural similarity comparison mechanisms, and/or pattern recognition to generate the cognified data. The cognified data may be output by the one or more trained machine learning models.

The cognified data may provide a summary of the medical condition of the patient. A diagnosis of the patient may be generated based on the cognified data. The summary of the medical condition may include one or more insights not present in the unstructured data. The summary may identify gaps in the unstructured data, such as treatment gaps (e.g., should prescribe medication, should provide different medication, should change dosage of medication, etc.), risk gaps (e.g., the patient is at risk for cancer based on familial history and certain lifestyle behaviors), quality of care gaps (e.g., need to check-in with the patient more frequently), and so forth. The summary of the medical condition may include one or more conclusions, recommendations, complications, risks, statements, causes, symptoms, etc. pertaining to the medical condition. In some embodiments, the summary of the medical condition may indicate another medical condition that the medical condition can lead to. Accordingly, the cognified data represents intelligence, knowledge, and logic cognified from unstructured data.

In some embodiments, the cognified data may be reviewed by physicians and the physicians may provide feedback pertaining to whether or not the cognified data is accurate. Also, the physicians may provide feedback pertaining to whether or not the diagnosis generated using the cognified data is accurate. This feedback may be used to update the one or more machine learning models to improve their accuracy.

The AI engine 109 may include machine learning models that are trained to schedule appointments for users, recommend appointments to users, determine costs of services, manage documents for users, extract data from images, provide curated content tailored for users, estimate wait times, perform natural language searching of curated content, and so forth.

The cognitive agent 110 represents a set of instructions executing within the cognitive intelligence platform 102 that implement a client-facing component of the cognitive intelligence platform 102. The cognitive agent 110 may be referred to as the autonomous multipurpose application interchangeably herein. The cognitive agent 110 is an interface between the cognitive intelligence platform 102 and the user device 104. And in some embodiments, the cognitive agent 110 includes a conversation orchestrator 124 that determines pieces of communication that are presented to the user device 104 (and the user). When a user of the user device 104 interacts with the cognitive intelligence platform 102, the user interacts with the cognitive agent 110. In some embodiments, the user of the user device 104 may be a patient. The several references herein, to the cognitive agent 110 performing a method, can implicate actions performed by the critical thinking engine 108, which accesses data in the knowledge cloud 106 and the natural language database 122.

Various user interfaces may be provided to computing devices communicating with the cognitive agent 110 executing in the cognitive intelligence platform 102. The user interfaces may be presented in a standalone application executing on the devices or in a web browser as website pages. In some embodiments, the cognitive agent 110 may be installed on a device of the user, the service provider 112, and/or the facility 114. In some embodiments, the devices of the user, the service provider 112, and/or the facility 114 may communicate with cognitive intelligence platform 102 in a client-server architecture. In some embodiments, the cognitive agent 110 may be implemented as computer instructions as an application programming interface.

In various embodiments, the several computing devices executing within the cognitive intelligence platform are communicably coupled by way of a network/bus interface. Furthermore, the various components (e.g., the knowledge cloud 106, the critical thinking engine 108, and the cognitive agent 110), are communicably coupled by one or more inter-host communication protocols 118. In one example, the knowledge cloud 106 is implemented using a first computing device, the critical thinking engine 108 is implemented using a second computing device, and the cognitive agent 110 is implemented using a third computing device, where each of the computing devices are coupled by way of the inter-host communication protocol 118. Although in this example, the individual components are described as executing on separate computing devices this example is not meant to be limiting, the components can be implemented on the same computing device, or partially on the same computing device, without departing from the scope of this disclosure.

The user device 104 represents any form of a computing device, or network of computing devices, e.g., a personal computing device, a smart phone, a tablet, a wearable computing device, a notebook computer, a media player device, and a desktop computing device. The user device 104 includes a processor, at least one memory, and at least one storage. A user uses the user device 104 to input a given text posed in natural language (e.g., typed on a physical keyboard, spoken into a microphone, typed on a touch screen, or combinations thereof) and interacts with the cognitive intelligence platform 102, by way of the cognitive agent 110.

The architecture 100 includes a network 120 that communicatively couples various devices, including the cognitive intelligence platform 102 and the user device 104. The network 120 can include local area network (LAN) and wide area networks (WAN). The network 102 can include wired technologies (e.g., Ethernet®) and wireless technologies (e.g., Wi-Fi®, code division multiple access (CDMA), global system for mobile (GSM), universal mobile telephone service (UMTS), Bluetooth®, and ZigBee®. For example, the user device 104 can use a wired connection or a wireless technology (e.g., Wi-Fi®) to transmit and receive data over the network 120.

Still referring to FIG. 1, the knowledge cloud 106 is configured to receive data from various sources and entities and integrate the data in a database. An example source that provides data to the knowledge could 106 is the service provider 112, an entity that provides a type of service to a user. For example, the service provider 112 can be a health service provider (e.g., a doctor's office, a physical therapist's office, a nurse's office, or a clinical social worker's office), and a financial service provider (e.g., an accountant's office). For purposes of this discussion, the cognitive intelligence platform 102 provides services in the health industry, thus the examples discussed herein are associated with the health industry. However, any service industry can benefit from the disclosure herein, and thus the examples associated with the health industry are not meant to be limiting.

Throughout the course of a relationship between the service provider 112 and a user (e.g., the service provider 112 provides healthcare to a patient), the service provider 112 collects and generates data associated with the patient or the user, including health records that include doctor's notes about the patient and prescriptions, billing records, and insurance records. The service provider 112, using a computing device (e.g., a desktop computer or a tablet), provides the data associated with the user to the cognitive intelligence platform 102, and more specifically the knowledge cloud 106.

Another example source that provides data to the knowledge cloud 106 is the facility 114. The facility 114 represents a location owned, operated, or associated with any entity including the service provider 112. As used herein, an entity represents an individual or a collective with a distinct and independent existence. An entity can be legally recognized (e.g., a sole proprietorship, a partnership, a corporation) or less formally recognized in a community. For example, the entity can include a company that owns or operates a gym (facility). Additional examples of the facility 114 include, but is not limited to, a hospital, a trauma center, a clinic, a dentist's office, a pharmacy, a store (including brick and mortar stores and online retailers), an out-patient care center, a specialized care center, a birthing center, a gym, a cafeteria, and a psychiatric care center.

As the facility 114 represents a large number of types of locations, for purposes of this discussion and to orient the reader by way of example, the facility 114 represents the doctor's office or a gym. The facility 114 generates additional data associated with the user such as appointment times, an attendance record (e.g., how often the user goes to the gym), a medical record, a billing record, a purchase record, an order history, and an insurance record. The facility 114, using a computing device (e.g., a desktop computer or a tablet), provides the data associated with the user to the cognitive intelligence platform 102, and more specifically the knowledge cloud 106.

An additional example source that provides data to the knowledge cloud 106 is the microsurvey 116. The microsurvey 116 represents a tool created by the cognitive intelligence platform 102 that enables the knowledge cloud 106 to collect additional data associated with the user. The microsurvey 116 is originally provided by the cognitive intelligence platform 102 (by way of the cognitive agent 110) and the user provides data responsive to the microsurvey 116 using the user device 104. Additional details of the microsurvey 116 are described below.

Yet another example source that provides data to the knowledge cloud 106, is the cognitive intelligence platform 102, itself. In order to address the care needs and well-being of the user, the cognitive intelligence platform 102 collects, analyzes, and processes information from the user, healthcare providers, and other eco-system participants, and consolidates and integrates the information into knowledge. For example, clinical-based evidence and guidelines may be obtained by the cognitive intelligence platform 102 and used as knowledge. The knowledge can be shared with the user and stored in the knowledge cloud 106.

In various embodiments, the computing devices used by the service provider 112 and the facility 114 are communicatively coupled to the cognitive intelligence platform 102, by way of the network 120. While data is used individually by various entities including: a hospital, practice group, facility, or provider, the data is less frequently integrated and seamlessly shared between the various entities in the current art. The cognitive intelligence platform 102 provides a solution that integrates data from the various entities. That is, the cognitive intelligence platform 102 ingests, processes, and disseminates data and knowledge in an accessible fashion, where the reason for a particular answer or dissemination of data is accessible by a user.

In particular, the cognitive intelligence platform 102 (e.g., by way of the cognitive agent 110 interacting with the user) holistically manages and executes a health plan for durational care and wellness of the user (e.g., a patient or consumer). The health plan includes various aspects of durational management that is coordinated through a care continuum.

The cognitive agent 110 can implement various personas that are customizable. For example, the personas can include knowledgeable (sage), advocate (coach), and witty friend (jester). And in various embodiments, the cognitive agent 110 persists with a user across various interactions (e.g., conversations streams), instead of being transactional or transient. Thus, the cognitive agent 110 engages in dynamic conversations with the user, where the cognitive intelligence platform 102 continuously deciphers topics that a user wants to talk about. The cognitive intelligence platform 102 has relevant conversations with the user by ascertaining topics of interest from a given text posed in a natural language input by the user. Additionally the cognitive agent 110 connects the user to healthcare service providers, hyperlocal health communities, and a variety of services and tools/devices, based on an assessed interest of the user.

As the cognitive agent 110 persists with the user, the cognitive agent 110 can also act as a coach and advocate while delivering pieces of information to the user based on tonal knowledge, human-like empathies, and motivational dialog within a respective conversational stream, where the conversational stream is a technical discussion focused on a specific topic. Overall, in response to a question—e.g., posed by the user in natural language—the cognitive intelligence platform 102 consumes data from and related to the user and computes an answer. The answer is generated using a rationale that makes use of common sense knowledge, domain knowledge, evidence-based medicine guidelines, clinical ontologies, and curated medical advice. Thus, the content displayed by the cognitive intelligence platform 102 (by way of the cognitive agent 110) is customized based on the language used to communicate with the user, as well as factors such as a tone, goal, and depth of topic to be discussed.

Overall, the cognitive intelligence platform 102 is accessible to a user, a hospital system, and physician. Additionally, the cognitive intelligence platform 102 is accessible to paying entities interested in user behavior—e.g., the outcome of physician-consumer interactions in the context of disease or the progress of risk management. Additionally, entities that provides specialized services such as tests, therapies, and clinical processes that need risk based interactions can also receive filtered leads from the cognitive intelligence platform 102 for potential clients.

Conversational Analysis

In various embodiments, the cognitive intelligence platform 102 is configured to perform conversational analysis in a general setting. The topics covered in the general setting is driven by the combination of agents (e.g., cognitive agent 110) selected by a user. In some embodiments, the cognitive intelligence platform 102 uses conversational analysis to identify the intent of the user (e.g., find data, ask a question, search for facts, find references, and find products) and a respective micro-theory in which the intent is logical.

For example, the cognitive intelligence platform 102 applies conversational analysis to decode what the user is asking or stated, where the question or statement is in free form language (e.g., natural language). Prior to determining and sharing knowledge (e.g., with the user or the knowledge cloud 106), using conversational analysis, the cognitive intelligence platform 102 identifies an intent of the user and overall conversational focus.

The cognitive intelligence platform 102 responds to a statement or question according to the conversational focus and steers away from another detected conversational focus so as to focus on a goal defined by the cognitive agent 110. Given an example statement of a user, “I want to fly out tomorrow,” the cognitive intelligence platform 102 uses conversational analysis to determine an intent of the statement. Is the user aspiring to be bird-like or does he want to travel? In the former case, the micro-theory is that of human emotions whereas in the latter case, the micro-theory is the world of travel. Answers are provided to the statement depending on the micro-theory in which the intent logically falls.

The cognitive intelligence platform 102 utilize a combination of linguistics, artificial intelligence, and decision trees to decode what a user is asking or stating. The discussion includes methods and system design considerations and results from an existing embodiment. Additional details related to conversational analysis are discussed next.

Analyzing Conversational Context as Part of Conversational Analysis

For purposes of this discussion, the concept of analyzing conversational context as part of conversational analysis is now described. To analyze conversational context, the following steps are taken: 1) obtain text (e.g., receive a question) and perform translations; 2) understand concepts, entities, intents, and micro-theory; 3) relate and search; 4) ascertain the existence of related concepts; 5) logically frame concepts or needs; 6) understand the questions that can be answered from available data; and 7) answer the question. Each of the foregoing steps is discussed next, in turn.

Step 1: Obtain Text/Question and Perform Translations

In various embodiments, the cognitive intelligence platform 102 (FIG. 1) receives a text or question and performs translations as appropriate. The cognitive intelligence platform 102 supports various methods of input including text received from a touch interface (e.g., options presented in a microsurvey), text input through a microphone (e.g., words spoken into the user device), and text typed on a keyboard or on a graphical user interface. Additionally, the cognitive intelligence platform 102 supports multiple languages and auto translation (e.g., from English to Traditional/Simplified Chinese or vice versa).

The example text below is used to described methods in accordance with various embodiments herein:
“One day in January 1913. G. H. Hardy, a famous Cambridge University mathematician received a letter from an Indian named Srinivasa Ramanujan asking him for his opinion of 120 mathematical theorems that Ramanujan said he had discovered. To Hardy, many of the theorems made no sense. Of the others, one or two were already well-known. Ramanujan must be some kind of trickplayer, Hardy decided, and put the letter aside. But all that day the letter kept hanging round Hardy. Might there by something in those wild-looking theorems?
That evening Hardy invited another brilliant Cambridge mathematician, J. E. Littlewood, and the two men set out to assess the Indian's worth. That incident was a turning point in the history of mathematics.
At the time, Ramanujan was an obscure Madras Port Trust clerk. A little more than a year later, he was at Cambridge University, and beginning to be recognized as one of the most amazing mathematicians the world has ever known. Though he died in 1920, much of his work was so far in advance of his time that only in recent years is it beginning to be properly understood.
Indeed, his results are helping solve today's problems in computer science and physics, problems that he could have had no notion of.
For Indians, moreover, Ramanujan has a special significance. Ramanujan, through born in poor and ill-paid accountant's family 100 years ago, has inspired many Indians to adopt mathematics as career.
Much of Ramanujan's work is in number theory, a branch of mathematics that deals with the subtle laws and relationships that govern numbers. Mathematicians describe his results as elegant and beautiful but they are much too complex to be appreciated by laymen.
His life, though, is full of drama and sorrow. It is one of the great romantic stories of mathematics, a distressing reminder that genius can surface and rise in the most unpromising circumstances.”

The cognitive intelligence platform 102 analyzes the example text above to detect structural elements within the example text (e.g., paragraphs, sentences, and phrases). In some embodiments, the example text is compared to other sources of text such as dictionaries, and other general fact databases (e.g., Wikipedia) to detect synonyms and common phrases present within the example text.

Step 2: Understand Concept, Entity, Intent, and Micro-Theory

In step 2, the cognitive intelligence platform 102 parses the text to ascertain concepts, entities, intents, and micro-theories. An example output after the cognitive intelligence platform 102 initially parses the text is shown below, where concepts, and entities are shown in bold.
“One day in January 1913. G. H. Hardy, a famous Cambridge University mathematician received a letter from an Indian named Srinivasa Ramanujan asking him for his opinion of 120 mathematical theorems that Ramanujan said he had discovered. To Hardy, many of the theorems made no sense. Of the others, one or two were already well-known. Ramanujan must be some kind of trickplayer, Hardy decided, and put the letter aside. But all that day the letter kept hanging round Hardy. Might there by something in those wild-looking theorems?
That evening Hardy invited another brilliant Cambridge mathematician, J. E. Littlewood, and the two men set out to assess the Indian's worth. That incident was a turning point in the history of mathematics.
At the time, Ramanujan was an obscure Madras Port Trust clerk. A little more than a year later, he was at Cambridge University, and beginning to be recognized as one of the most amazing mathematicians the world has ever known. Though he died in 1920, much of his work was so far in advance of his time that only in recent years is it beginning to be properly understood.
Indeed, his results are helping solve today's problems in computer science and physics, problems that he could have had no notion of.
For Indians, moreover, Ramanujan has a special significance. Ramanujan, through born in poor and ill-paid accountant's family 100 years ago, has inspired many Indians to adopt mathematics as career.
Much of Ramanujan's work is in number theory, a branch of mathematics that deals with the subtle laws and relationships that govern numbers. Mathematicians describe his results as elegant and beautiful but they are much too complex to be appreciated by laymen.
His life, though, is full of drama and sorrow. It is one of the great romantic stories of mathematics, a distressing reminder that genius can surface and rise in the most unpromising circumstances.”

For example, the cognitive intelligence platform 102 ascertains that Cambridge is a university—which is a full understanding of the concept. The cognitive intelligence platform (e.g., the cognitive agent 110) understands what humans do in Cambridge, and an example is described below in which the cognitive intelligence platform 102 performs steps to understand a concept.

For example, in the context of the above example, the cognitive agent 110 understands the following concepts and relationships:
Cambridge employed John Edensor Littlewood (1)
Cambridge has the position Ramanujan's position at Cambridge University (2)
Cambridge employed G. H. Hardy. (3)

The cognitive agent 110 also assimilates other understandings to enhance the concepts, such as:
Cambridge has Trinity College as a suborganization. (4)
Cambridge is located in Cambridge. (5)
Alan Turing is previously enrolled at Cambridge. (6)
Stephen Hawking attended Cambridge. (7)

The statements (1)-(7) are not picked at random. Instead the cognitive agent 110 dynamically constructs the statements (1)-(7) from logic or logical inferences based on the example text above. Formally, the example statements (1)-(7) are captured as follows:
(#$subOrganizations #$UniversityOfCambridge #$TrinityCollege-Cambridge-England) (8)
(#$placeInCity #$UniversityOfCambridge #$Cityof CambridgeEngland) (9)
(#$schooling #$AlanTuring #$UniversityOfCambridge #$PreviouslyEnrolled)(10)
(#$hasAlumni #$UniversityOfCambridge #$StephenHawking) (11)

Step 3: Relate and Search

Next, in step 3, the cognitive agent 110 relates various entities and topics and follows the progression of topics in the example text. Relating includes the cognitive agent 110 understanding the different instances of Hardy are all the same person, and the instances of Hardy are different from the instances of Littlewood. The cognitive agent 110 also understands that the instances Hardy and Littlewood share some similarities—e.g., both are mathematicians and they did some work together at Cambridge on Number Theory. The ability to track this across the example text is referred to as following the topic progression with a context.

Step 4: Ascertain the Existence of Related Concepts

Next, in Step 4, the cognitive agent 110 asserts non-existent concepts or relations to form new knowledge. Step 4 is an optional step for analyzing conversational context. Step 4 enhances the degree to which relationships are understood or different parts of the example text are understood together. If two concepts appear to be separate—e.g., a relationship cannot be graphically drawn or logically expressed between enough sets of concepts—there is a barrier to understanding. The barriers are overcome by expressing additional relationships. The additional relationships can be discovered using strategies like adding common sense or general knowledge sources (e.g., using the common sense data 208) or adding in other sources including a lexical variant database, a dictionary, and a thesaurus.

One example of concept progression from the example text is as follows: the cognitive agent 110 ascertains the phrase “theorems that Ramanujan said he had discovered” is related to the phrase “his results”, which is related to “Ramanujan's work is in number theory, a branch of mathematics that deals with the subtle laws and relationships that govern numbers.”

Step 5: Logically Frame Concepts or Needs

In Step 5, the cognitive agent 110 determines missing parameters—which can include for example, missing entities, missing elements, and missing nodes—in the logical framework (e.g., with a respective micro-theory). The cognitive agent 110 determines sources of data that can inform the missing parameters. Step 5 can also include the cognitive agent 110 adding common sense reasoning and finding logical paths to solutions.

With regards to the example text, some common sense concepts include:
Mathematicians develop Theorems. (12)
Theorems are hard to comprehend. (13)
Interpretations are not apparent for years. (14)
Applications are developed over time. (15)
Mathematicians collaborate and assess work. (16)

With regards to the example text, some passage concepts include:
Ramanujan did Theorems in Early 20th Century. (17)
Hardy assessed Ramanujan's Theorems. (18)
Hardy collaborated with Littlewood. (19)
Hardy and Littlewood assessed Ramanujan's work (20)
   Within the micro-theory of the passage analysis, the cognitive agent 110 understands and catalogs available paths to answer questions. In Step 5, the cognitive agent 110 makes the case that the concepts (12)-(20) are expressed together.

Step 6: Understand the Questions that can be Answered from Available Data

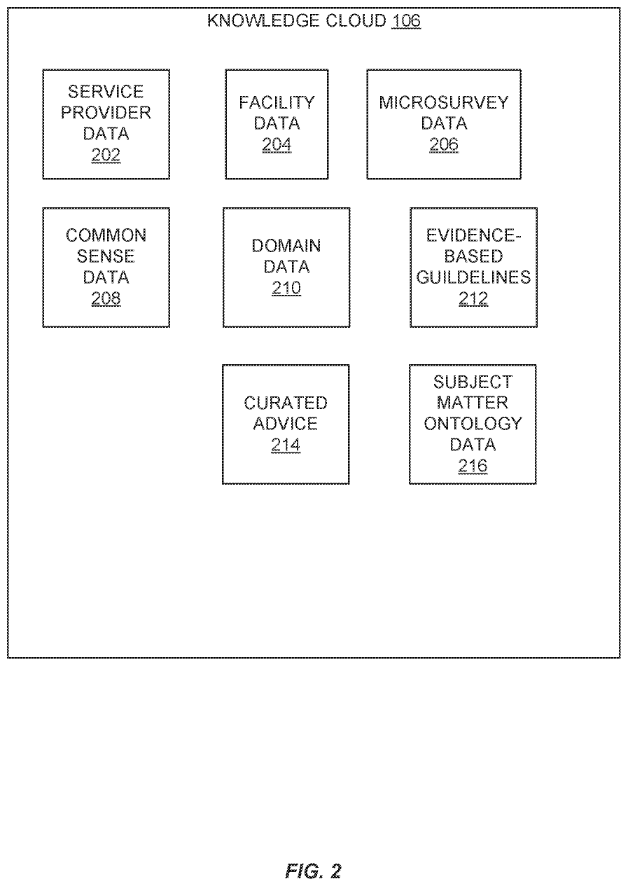

In Step 6, the cognitive agent 110 parses sub-intents and entities. Given the example text, the following questions are answerable from the cognitive agent's developed understanding of the example text, where the understanding was developed using information and context ascertained from the example text as well as the common sense data 208 (FIG. 2):
What situation causally contributed to Ramanujan's position at Cambridge? (21)
Does the author of the passage regret that Ramanujan died prematurely? (22)
Does the author of the passage believe that Ramanujan is a mathematical genius?(23) Based on the information that is understood by the cognitive agent 110, the questions (21)-(23) can be answered.

By using an exploration method such as random walks, the cognitive agent 110 makes a determination as the paths that are plausible and reachable with the context (e.g., micro-theory) of the example text. Upon explorations, the cognitive agent 110 catalogs a set of meaningful questions. The set of meaningful questions are not asked, but instead explored based on the cognitive agent's understanding of the example text.

Given the example text, an example of exploration that yields a positive result is: “a situation X that caused Ramanujan's position.” In contrast, an example of exploration that causes irrelevant results is: “a situation Y that caused Cambridge.” The cognitive agent 110 is able to deduce that the latter exploration is meaningless, in the context of a micro-theory, because situations do not cause universities. Thus the cognitive agent 110 is able to deduce, there are no answers to Y, but there are answers to X.

Step 7: Answer the Question

In Step 7, the cognitive agent 110 provides a precise answer to a question. For an example question such as: “What situation causally contributed to Ramanujan's position at Cambridge?” the cognitive agent 110 generates a precise answer using the example reasoning:
HardyandLittlewoodsEvaluatingOfRamanujansWork (24)
HardyBeliefThatRamanujanIsAnExpertInMathematics (25)
HardysBeliefThatRamanujanIsAnExpertInMathematicsAndAGenius (26) In order to generate the above reasoning statements (24)-(26), the cognitive agent 110 utilizes a solver or prover in the context of the example text's micro-theory—and associated facts, logical entities, relations, and assertions. As an additional example, the cognitive agent 110 uses a reasoning library that is optimized for drawing the example conclusions above within the fact, knowledge, and inference space (e.g., work space) that the cognitive agent 110 maintains.

By implementing the steps 1-7, the cognitive agent 110 analyzes conversational context. The described method for analyzing conversation context can also be used for recommending items in conversations streams. A conversational stream is defined herein as a technical discussion focused on specific topics. As related to described examples herein, the specific topics relate to health (e.g., diabetes). Throughout the lifetime of a conversational stream, a cognitive agent 110 collect information over may channels such as chat, voice, specialized applications, web browsers, contact centers, and the like.

By implementing the methods to analyze conversational context, the cognitive agent 110 can recommend a variety of topics and items throughout the lifetime of the conversational stream. Examples of items that can be recommended by the cognitive agent 110 include: surveys, topics of interest, local events, devices or gadgets, dynamically adapted health assessments, nutritional tips, reminders from a health events calendar, and the like.

Accordingly, the cognitive intelligence platform 102 provides a platform that codifies and takes into consideration a set of allowed actions and a set of desired outcomes. The cognitive intelligence platform 102 relates actions, the sequences of subsequent actions (and reactions), desired sub-outcomes, and outcomes, in a way that is transparent and logical (e.g., explainable). The cognitive intelligence platform 102 can plot a next best action sequence and a planning basis (e.g., health care plan template, or a financial goal achievement template), also in a manner that is explainable. The cognitive intelligence platform 102 can utilize a critical thinking engine 108 and a natural language database 122 (e.g., a linguistics and natural language understanding system) to relate conversation material to actions.

For purposes of this discussion, several examples are discussed in which conversational analysis is applied within the field of durational and whole-health management for a user. The discussed embodiments holistically address the care needs and well-being of the user during the course of his life. The methods and systems described herein can also be used in fields outside of whole-health management, including: phone companies that benefits from a cognitive agent; hospital systems or physicians groups that want to coach and educate patients; entities interested in user behavior and the outcome of physician-consumer interactions in terms of a progress of disease or risk management; entities that provide specialized services (e.g., test, therapies, clinical processes) to filter leads; and sellers, merchants, stores and big box retailers that want to understand which product to sell.

In addition, the conversational analysis may include cognifying the text input by the user. For example, if the user states (e.g., text, voice) they have various symptoms, the cognification techniques disclosed herein may be performed to construct cognified data using the text input. The user may input text specifying that they have a level of 5.7 mmol/L blood sugar. The cognitive intelligence platform 102 may cognify the text to output that the level of blood sugar is within acceptable limits, and that blood sugar testing was used to measure the blood sugar level. In some embodiments, the cognification techniques may be performed to generate a diagnosis of a medical condition of the patient. Further, the cognitive intelligence platform 102 may provide information to the user pertaining to the medical condition at a regulated pace.

FIG. 2 shows additional details of a knowledge cloud, in accordance with various embodiments. In particular, FIG. 2 illustrates various types of data received from various sources, including service provider data 202, facility data 204, microsurvey data 206, commonsense data 208, domain data 210, evidence-based guidelines 212, subject matter ontology data 214, and curated advice 216. The types of data represented by the service provider data 202 and the facility data 204 include any type of data generated by the service provider 112 and the facility 114, and the above examples are not meant to be limiting. Thus, the example types of data are not meant to be limiting and other types of data can also be stored within the knowledge cloud 106 without departing from the scope of this disclosure.

The service provider data 202 is data provided by the service provider 112 (described in FIG. 1) and the facility data 204 is data provided by the facility 114 (described in FIG. 1). For example, the service provider data 202 includes medical records of a respective patient of a service provider 112 that is a doctor. In another example, the facility data 204 includes an attendance record of the respective patient, where the facility 114 is a gym. The microsurvey data 206 is data provided by the user device 104 responsive to questions presented in the microsurvey 116 (FIG. 1).

Common sense data 208 is data that has been identified as “common sense”, and can include rules that govern a respective concept and used as glue to understand other concepts.

Domain data 210 is data that is specific to a certain domain or subject area. The source of the domain data 210 can include digital libraries. In the healthcare industry, for example, the domain data 210 can include data specific to the various specialties within healthcare such as, obstetrics, anesthesiology, and dermatology, to name a few examples. In the example described herein, the evidence-based guidelines 212 include systematically developed statements to assist practitioner and patient decisions about appropriate health care for specific clinical circumstances.

Curated advice 214 includes advice from experts in a subject matter. The curated advice 214 can include peer-reviewed subject matter, and expert opinions. Subject matter ontology data 216 includes a set of concepts and categories in a subject matter or domain, where the set of concepts and categories capture properties and relationships between the concepts and categories.

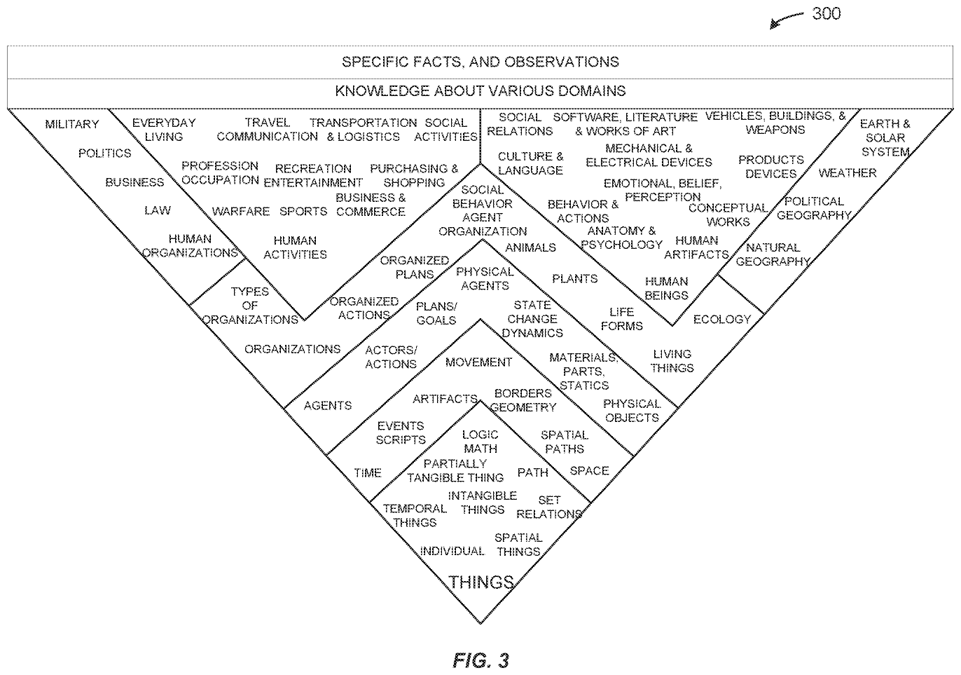

In particular, FIG. 3 illustrates an example subject matter ontology 300 that is included as part of the subject matter ontology data 216.

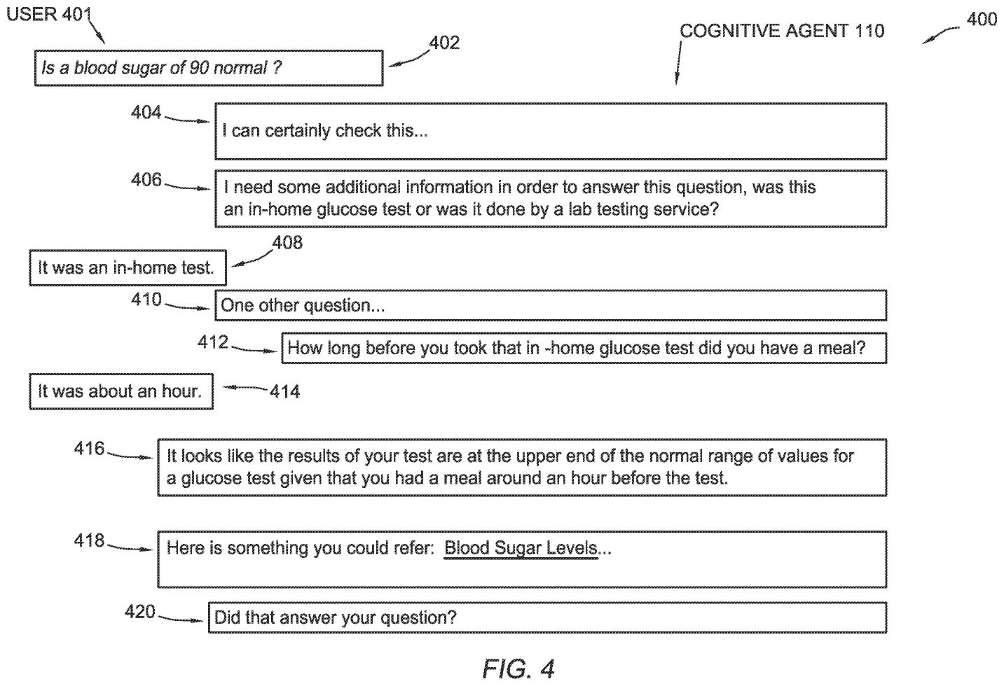

FIG. 4 illustrates aspects of a conversation 400 between a user and the cognitive intelligence platform 102, and more specifically the cognitive agent 110. For purposes of this discussion, the user 401 is a patient of the service provider 112. The user interacts with the cognitive agent 110 using a computing device, a smart phone, or any other device configured to communicate with the cognitive agent 110 (e.g., the user device 104 in FIG. 1). The user can enter text into the device using any known means of input including a keyboard, a touchscreen, and a microphone. The conversation 400 represents an example graphical user interface (GUI) presented to the user 401 on a screen of his computing device.

Initially, the user asks a general question, which is treated by the cognitive agent 110 as an “originating question.” The originating question is classified into any number of potential questions (“pursuable questions”) that are pursued during the course of a subsequent conversation. In some embodiments, the pursuable questions are identified based on a subject matter domain or goal. In some embodiments, classification techniques are used to analyze language (e.g., such as those outlined in HPS ID20180901-01_method for conversational analysis). Any known text classification technique can be used to analyze language and the originating question. For example, in line 402, the user enters an originating question about a subject matter (e.g., blood sugar) such as: “Is a blood sugar of 90 normal”?

In response to receiving an originating question, the cognitive intelligence platform 102 (e.g., the cognitive agent 110 operating in conjunction with the critical thinking engine 108) performs a first round of analysis (e.g., which includes conversational analysis) of the originating question and, in response to the first round of analysis, creates a workspace and determines a first set of follow up questions.

In various embodiments, the cognitive agent 110 may go through several rounds of analysis executing within the workspace, where a round of analysis includes: identifying parameters, retrieving answers, and consolidating the answers. The created workspace can represent a space where the cognitive agent 110 gathers data and information during the processes of answering the originating question. In various embodiments, each originating question corresponds to a respective workspace. The conversation orchestrator 124 can assess data present within the workspace and query the cognitive agent 110 to determine if additional data or analysis should be performed.

In particular, the first round of analysis is performed at different levels, including analyzing natural language of the text, and analyzing what specifically is being asked about the subject matter (e.g., analyzing conversational context). The first round of analysis is not based solely on a subject matter category within which the originating question is classified. For example, the cognitive intelligence platform 102 does not simply retrieve a predefined list of questions in response to a question that falls within a particular subject matter, e.g., blood sugar. That is, the cognitive intelligence platform 102 does not provide the same list of questions for all questions related to the particular subject matter. Instead, for example, the cognitive intelligence platform 102 creates dynamically formulated questions, curated based on the first round of analysis of the originating question.

In particular, during the first round of analysis, the cognitive agent 110 parses aspects of the originating question into associated parameters. The parameters represent variables useful for answering the originating question. For example, the question “is a blood sugar of 90 normal” may be parsed and associated parameters may include, an age of the inquirer, the source of the value 90 (e.g., in home test or a clinical test), a weight of the inquirer, and a digestive state of the user when the test was taken (e.g., fasting or recently eaten). The parameters identify possible variables that can impact, inform, or direct an answer to the originating question.

For purposes of the example illustrated in FIG. 4, in the first round of analysis, the cognitive intelligence platform 102 inserts each parameter into the workspace associated with the originating question (line 402). Additionally, based on the identified parameters, the cognitive intelligence platform 102 identifies a customized set of follow up questions (“a first set of follow-up questions). The cognitive intelligence platform 102 inserts first set of follow-up questions in the workspace associated with the originating question.

The follow up questions are based on the identified parameters, which in turn are based on the specifics of the originating question (e.g., related to an identified micro-theory). Thus the first set of follow-up questions identified in response to, if a blood sugar is normal, will be different from a second set of follow up questions identified in response to a question about how to maintain a steady blood sugar.

After identifying the first set of follow up questions, in this example first round of analysis, the cognitive intelligence platform 102 determines which follow up question can be answered using available data and which follow-up question to present to the user. As described over the next few paragraphs, eventually, the first set of follow-up questions is reduced to a subset (“a second set of follow-up questions”) that includes the follow-up questions to present to the user.

In various embodiments, available data is sourced from various locations, including a user account, the knowledge cloud 106, and other sources. Other sources can include a service that supplies identifying information of the user, where the information can include demographics or other characteristics of the user (e.g., a medical condition, a lifestyle). For example, the service can include a doctor's office or a physical therapist's office.

Another example of available data includes the user account. For example, the cognitive intelligence platform 102 determines if the user asking the originating question, is identified. A user can be identified if the user is logged into an account associated with the cognitive intelligence platform 102. User information from the account is a source of available data. The available data is inserted into the workspace of the cognitive agent 110 as a first data.

Another example of available data includes the data stored within the knowledge cloud 106. For example, the available data includes the service provider data 202 (FIG. 2), the facility data 204, the microsurvey data 206, the common sense data 208, the domain data 210, the evidence-based guidelines 212, the curated advice 214, and the subject matter ontology data 216. Additionally data stored within the knowledge cloud 106 includes data generated by the cognitive intelligence platform 102, itself.

Follow up questions presented to the user (the second set of follow-up questions) are asked using natural language and are specifically formulated (“dynamically formulated question”) to elicit a response that will inform or fulfill an identified parameter. Each dynamically formulated question can target one parameter at a time. When answers are received from the user in response to a dynamically formulated question, the cognitive intelligence platform 102 inserts the answer into the workspace. In some embodiments, each of the answers received from the user and in response to a dynamically formulated question, is stored in a list of facts. Thus the list of facts include information specifically received from the user, and the list of facts is referred to herein as the second data.

With regards to the second set of follow-up questions (or any set of follow-up questions), the cognitive intelligence platform 102 calculates a relevance index, where the relevance index provides a ranking of the questions in the second set of follow-up questions. The ranking provides values indicative of how relevant a respective follow-up question is to the originating question. To calculate the relevance index, the cognitive intelligence platform 102 can use conversations analysis techniques described in HPS ID20180901-01_method. In some embodiments, the first set or second set of follow up questions is presented to the user in the form of the microsurvey 116.

In this first round of analysis, the cognitive intelligence platform 102 consolidates the first and second data in the workspace and determines if additional parameters need to be identified, or if sufficient information is present in the workspace to answer the originating question. In some embodiments, the cognitive agent 110 (FIG. 1) assesses the data in the workspace and queries the cognitive agent 110 to determine if the cognitive agent 110 needs more data in order to answer the originating question. The conversation orchestrator 124 executes as an interface

For a complex originating question, the cognitive intelligence platform 102 can go through several rounds of analysis. For example, in a first round of analysis the cognitive intelligence platform 102 parses the originating question. In a subsequent round of analysis, the cognitive intelligence platform 102 can create a sub question, which is subsequently parsed into parameters in the subsequent round of analysis. The cognitive intelligence platform 102 is smart enough to figure out when all information is present to answer an originating question without explicitly programming or pre-programming the sequence of parameters that need to be asked about.

In some embodiments, the cognitive agent 110 is configured to process two or more conflicting pieces of information or streams of logic. That is, the cognitive agent 110, for a given originating question can create a first chain of logic and a second chain of logic that leads to different answers. The cognitive agent 110 has the capability to assess each chain of logic and provide only one answer. That is, the cognitive agent 110 has the ability to process conflicting information received during a round of analysis.

Additionally, at any given time, the cognitive agent 110 has the ability to share its reasoning (chain of logic) to the user. If the user does not agree with an aspect of the reasoning, the user can provide that feedback which results in affecting change in a way the critical thinking engine 108 analyzed future questions and problems.

Subsequent to determining enough information is present in the workspace to answer the originating question, the cognitive agent 110 answers the question, and additionally can suggest a recommendation or a recommendation (e.g., line 418). The cognitive agent 110 suggests the reference or the recommendation based on the context and questions being discussed in the conversation (e.g., conversation 400). The reference or recommendation serves as additional handout material to the user and is provided for informational purposes. The reference or recommendation often educates the user about the overall topic related to the originating question.

In the example illustrated in FIG. 4, in response to receiving the originating questions (line 402), the cognitive intelligence platform 102 (e.g., the cognitive agent 110 in conjunction with the critical thinking engine 108) parses the originating question to determine at least one parameter: location. The cognitive intelligence platform 102 categorizes this parameter, and a corresponding dynamically formulated question in the second set of follow-up questions. Accordingly, in lines 404 and 406, the cognitive agent 110 responds by notifying the user “I can certainly check this . . . ” and asking the dynamically formulated question “I need some additional information in order to answer this question, was this an in-home glucose test or was it done by a lab or testing service?”

The user 401 enters his answer in line 408: “It was an in-home test,” which the cognitive agent 110 further analyzes to determine additional parameters: e.g., a digestive state, where the additional parameter and a corresponding dynamically formulated question as an additional second set of follow-up questions. Accordingly, the cognitive agent 110 poses the additional dynamically formulated question in lines 410 and 412: “One other question . . . ” and “How long before you took that in-home glucose test did you have a meal?” The user provides additional information in response “it was about an hour” (line 414).

The cognitive agent 110 consolidates all the received responses using the critical thinking engine 108 and the knowledge cloud 106 and determines an answer to the initial question posed in line 402 and proceeds to follow up with a final question to verify the user's initial question was answered. For example, in line 416, the cognitive agent 110 responds: “It looks like the results of your test are at the upper end of the normal range of values for a glucose test given that you had a meal around an hour before the test.” The cognitive agent 110 provides additional information (e.g., provided as a link): “Here is something you could refer,” (line 418), and follows up with a question “Did that answer your question?” (line 420).

As described above, due to the natural language database 108, in various embodiments, the cognitive agent 110 is able to analyze and respond to questions and statements made by a user 401 in natural language. That is, the user 401 is not restricted to using certain phrases in order for the cognitive agent 110 to understand what a user 401 is saying. Any phrasing, similar to how the user would speak naturally can be input by the user and the cognitive agent 110 has the ability to understand the user.

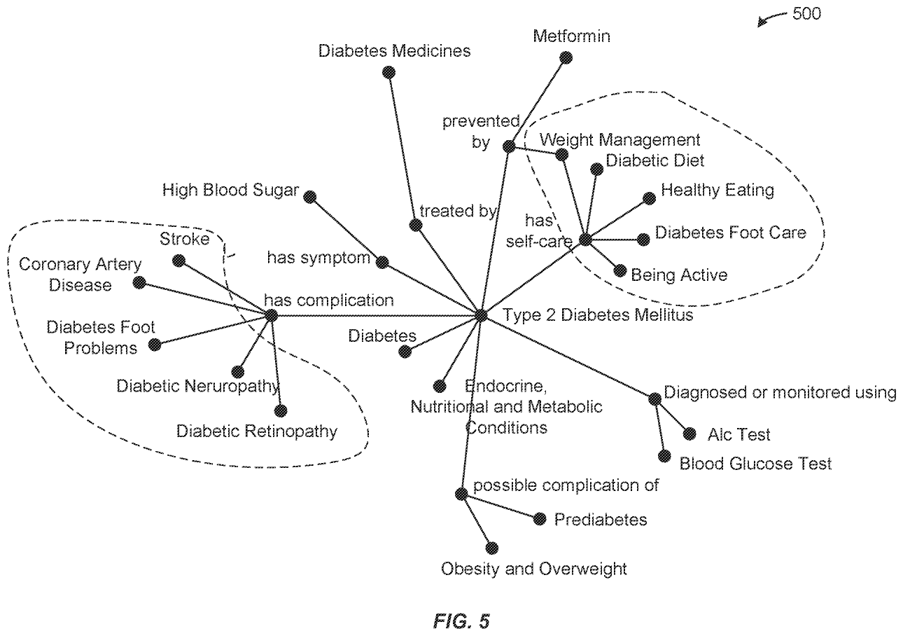

FIG. 5 illustrates a cognitive map or “knowledge graph” 500, in accordance with various embodiments. In particular, the knowledge graph represents a graph traversed by the cognitive intelligence platform 102, when assessing questions from a user with Type 2 diabetes. Individual nodes in the knowledge graph 500 represent a health artifact (health related information) or relationship (predicate) that is gleaned from direct interrogation or indirect interactions with the user (by way of the user device 104).

In one embodiment, the cognitive intelligence platform 102 identified parameters for an originating question based on a knowledge graph illustrated in FIG. 5. For example, the cognitive intelligence platform 102 parses the originating question to determine which parameters are present for the originating question. In some embodiments, the cognitive intelligence platform 102 infers the logical structure of the parameters by traversing the knowledge graph 500, and additionally, knowing the logical structure enables the cognitive agent 110 to formulate an explanation as to why the cognitive agent 110 is asking a particular dynamically formulated question.

In some embodiments, the individual elements or nodes are generated by the artificial intelligence engine based on input data (e.g., evidence-based guidelines, patient notes, clinical trials, physician research or the like). The artificial intelligence engine may parse the input data and construct the relationships between the health artifacts.

For example, a root node may be associated with a first health related information “Type 2 Diabetes Mellitus”, which is a name of a medical condition. In some embodiments, the root node may also be associated with a definition of the medical condition. An example predicate, “has symptom”, is represented by an individual node connected to the root node, and another health related information, “High Blood Sugar”, is represented by an individual node connected to the individual node representing the predicate. A logical structure may be represented by these three nodes, and the logical structure may indicate that “Type 2 Diabetes Mellitus has symptom High Blood Sugar”.

In some embodiments, the health related information may correspond to known facts, concepts, and/or any suitable health related information that are discovered or provided by a trusted source (e.g., a physician having a medical license and/or a certified/accredited healthcare organization), such as evidence-based guidelines, clinical trials, physician research, patient notes entered by physicians, and the like. The predicates may be part of a logical structure (e.g., sentence) such as a form of subject-predicate-direct object, subject-predicate-indirect object-direct object, subject-predicate-subject complement, or any suitable simple, compound, complex, and/or compound/complex logical structure. The subject may be a person, place, thing, health artifact, etc. The predicate may express an action or being within the logical structure and may be a verb, modifying words, phrases, and/or clauses. For example, one logical structure may be the subject-predicate-direct object form, such as “A has B” (where A is the subject and may be a noun or a health artifact, “has” is the predicate, and B is the direct object and may be a health artifact).

The various logical structures in the depicted knowledge graph may include the following: “Type 2 Diabetes Mellitus has symptom High Blood Sugar”; “Type 2 Diabetes Mellitus has complication Stroke”; “Type 2 Diabetes Mellitus has complication Coronary Artery Disease”; “Type 2 Diabetes Mellitus has complication Diabetes Foot Problems”; “Type 2 Diabetes Mellitus has complication Diabetic Neuropathy”; “Type 2 Diabetes Mellitus has complication Diabetic Retinopathy”; “Type 2 Diabetes Mellitus diagnosed or monitored using Blood Glucose Test”; just to name a few examples. It should be understood that there are other logical structures and represented in the knowledge graph 500.

In some embodiments, the information depicted in the knowledge graph may be represented as a matrix. The health artifacts may be represented as quantities and the predicates may be represented as expressions in a rectangular array in rows and columns of the matrix. The matrix may be treated as a single entity and manipulated according to particular rules.

The knowledge graph 500 or the matrix may be generated for each known medical condition and stored by the cognitive intelligence platform 102. The knowledge graphs and/or matrices may be updated continuously or on a periodic basis using subject data pertaining to the medical conditions received from the trusted sources. For example, additional clinical trials may lead to new discoveries about particular medical condition treatments, which may be used to update the knowledge graphs and/or matrices.

The knowledge graph 500 including the logical structures may be used to transform unstructured data (patient notes in an EMR entered by a physician) into cognified data. The cognified data may be used to generate a diagnosis of the patient. Also, the cognified data may be used to determine which information pertaining to the medical condition to provide to the patient and when to provide the information to the patient to improve the user experience using the computing device. The disclosed techniques may also save computing resources by providing the cognified data to the physician to review, improve diagnosis accuracy, and/or regulate the amount of information provided to the patient.

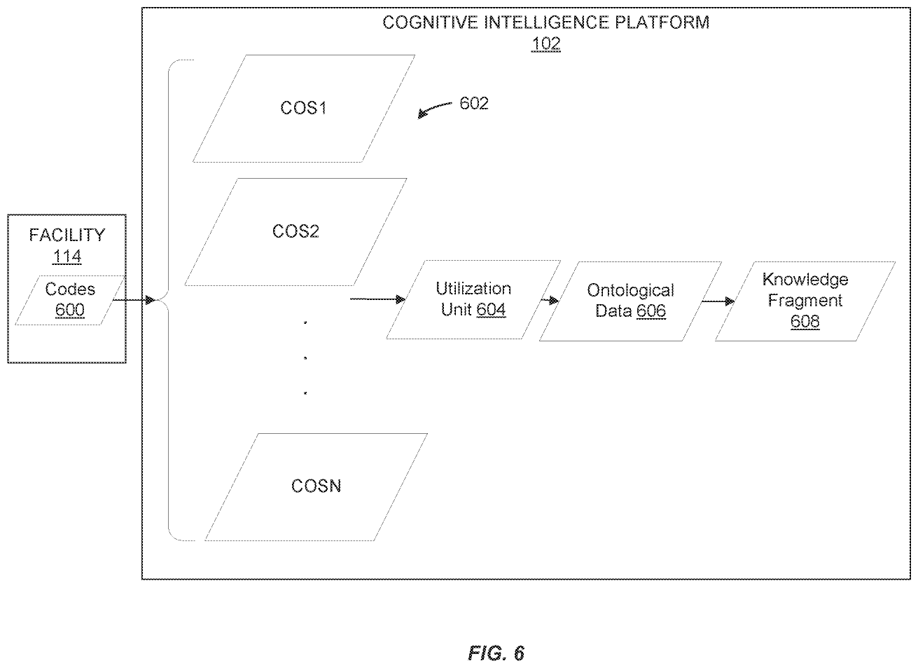

FIG. 6 shows an example block diagram performing mapping operations, in accordance with various embodiments. Any type of interaction, event, encounter, treatment, medical condition, and so forth pertaining to healthcare is represented by codes. There are a multitude of codes, such as International Classification of Diseases (ICD), Revenue Codes (RevCodes), Type of Service (TOS), Place of Service (POS), Healthcare Common Procedure Coding System, Current Procedural Terminology (CPT), and so forth. The ICD includes codes and classifications for conditions and diagnoses. In the United States, there are two types of ICD systems: (i) ICD-CM (Clinical Modification) that is used for diagnosis, and (ii) ICD-PCS (Procedure Coding System) that is used for inpatient hospital procedures. The ICD may be used to classify mortality and morbidity statistics, and may define diseases and allocate resources to provide care. ICD codes are alphanumeric designations given to every diagnosis, description of symptoms and cause of death attributed to human beings. ICD codes indicate signs, symptoms, diseases, conditions, and injuries to payers injuries, diseases, and conditions. These codes are used in conjunction with CPT (procedural) codes to record services rendered by a provider to a patient and is documented in the medical record and then reported to a payer (e.g., insurance provider) for reimbursement.

CPT codes are standard codes that are organized in three categories: (i) Category 1—five digit codes with descriptions that correspond to a service or procedure, (ii) Category 2—alphanumeric tracking codes for execution measurement, and (iii) Category 3—provisional codes for new and developing technology, procedures, and services. CPT codes provide a uniform data set that can be used to describe medical, surgical, and diagnostic services rendered to patients. CPT codes and ICD codes may be submitted on claims forms to insurance providers and the forms are used to determine reimbursement to a provider that rendered the service and/or facility at which the service was rendered.

HCPCS is based on CPT. HCPCS codes are generally used for supplies and products that are not directly related to a physician, for example, ambulance services, drugs, and the like.

In the depicted example, a person may perform a service for a user at the facility 114. For example, in the healthcare industry, a medical personnel may perform a coronary artery bypass on a patient at a hospital. There are many codes 8500 that are used to describe the event. There may be a code to represent the patient being admitted for in-patient care, room and board, the coronary artery bypass graft (CABG) procedure, the coronary artery bypass procedure has further details represented by codes (e.g., a code that indicates a top portion of a certain artery was removed, a code that indicates a certain vein was taken from a certain leg, etc.). The codes associated with the procedure may be input in a claims form and/or an electronic medical records (EMR) system that is communicatively connected to the cognitive intelligence platform 102.

The codes 600 may be transmitted to the cognitive intelligence platform 102. Oftentimes, the codes 600 are not completely and/or accurately input by a person. There may be missing codes that make understanding what exactly occurred difficult. The disclosed techniques may map the codes using a taxonomy of data 602 to determine a utilization unit (e.g., a procedure that was performed, a service rendered, a condition the procedure was performed for, etc.). To that end, the cognitive intelligence platform 102 may store a taxonomy of data 602 that may be applied to anything that is experienced by a patient or performed on a patient at a healthcare facility 114.

The taxonomy of data 602 may be organized into any suitable number of levels. For example, one level may include a Category of Service 1 (COS1), another level may include a Category of Service 2 (COS2), and there may be any suitable number of levels until COSN. Each COS may include various different types of codes. For example, COS1 may include CPT codes, HCPCS codes, and/or RevCodes. COS2 may include the same or different types of codes, for example, COS2 may include POS codes, TOS codes, ICD codes, and so forth.

In some embodiments, the mapping may enable determining what event occurred, which may further enable determining what type of intervention to perform. An intervention may include messaging services including action instructions to a medical personnel, a patient, an administrator, or some combination thereof. An intervention may include dispatching an emergency service personnel to a location of the patient (e.g., determined based on geolocation data of a user device 104), calling the user device 104, or some combination thereof.

The codes 600 may be mapped to the taxonomy of data 602. For example, code 12345 may be mapped in COS1 to an in-patient admit at a hospital, code AS123 may be mapped in COS2 to a surgery, AS434 may be mapped to a child level of surgery in COS2 that represents a coronary artery bypass procedure, and so forth. Based on the mappings, the artificial-intelligence engine 109 may output a utilization unit 8504 (e.g., CABG).

Once the utilization unit 604 is determined, the utilization unit 604 may be mapped to ontological data 606. The ontological data 606 may be represented by a knowledge graph (e.g., knowledge graph 500) that pertains to the determined utilization unit 604. For example, the CABG utilization unit may be determined to be performed when a patient has coronary artery disease. Accordingly, ontological data 606 (e.g., knowledge graph) may be obtained for the coronary artery disease medical condition.

The ontological data 606 may be mapped to a knowledge fragment 608. In some embodiments, a knowledge fragment may refer to data representing a specific portion of the ontological data included in the knowledge graph of the medical condition. For example, the knowledge fragment may include a concept, an evidence-based guideline, a proven fact, or the like pertaining to the medical condition (e.g., “Coronary artery bypass grafting takes three to four months to fully recover”). In some embodiments, the knowledge fragment 608 may refer to an action instruction that is determined by comparing the knowledge graph of the medical condition to a patient graph specific to the patient and the medical condition and/or any other patient graph specific to the patient and other medical conditions of the patient. The patient graph specific to the medical condition (e.g., coronary artery disease) may indicate that the patient has performed certain interactions or encounters (e.g., events) with health artifacts in the knowledge graph for the medical condition, but not other health artifacts in the knowledge graph. Accordingly, the knowledge fragment 608 may include an action instruction for a medical personnel and/or a patient to perform an action pertaining to the interactions or encounters that have not been performed yet.

The knowledge fragment 608 may be transmitted to a computing device of a medical personnel for presentation on the computing device. In some embodiments, the knowledge fragment may be used to perform an intervention. For example, the knowledge fragment 608 may indicate that the patient is at high risk for contracting a disease and the intervention may include sending a medication alert to the computing device of the medical personnel to instruct the medical personnel to prescribe a certain medicine.

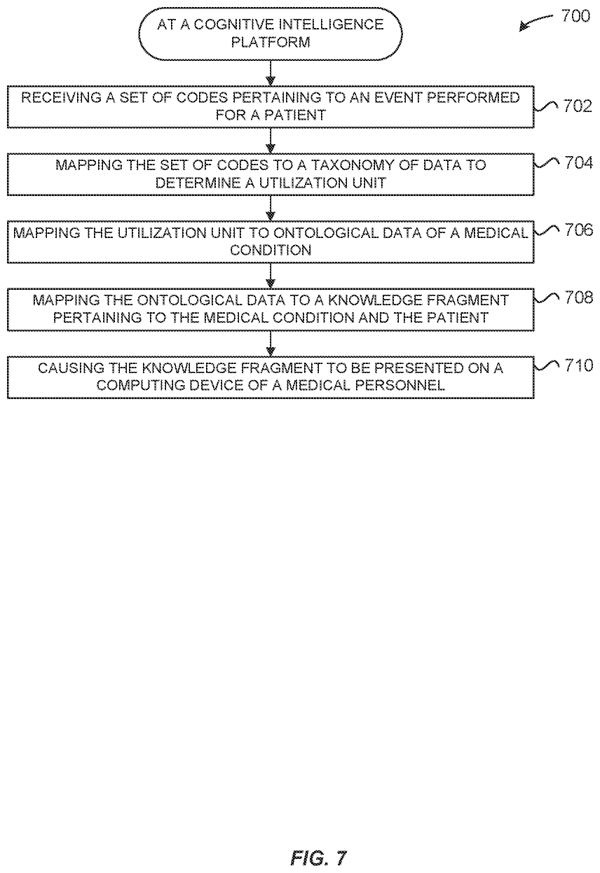

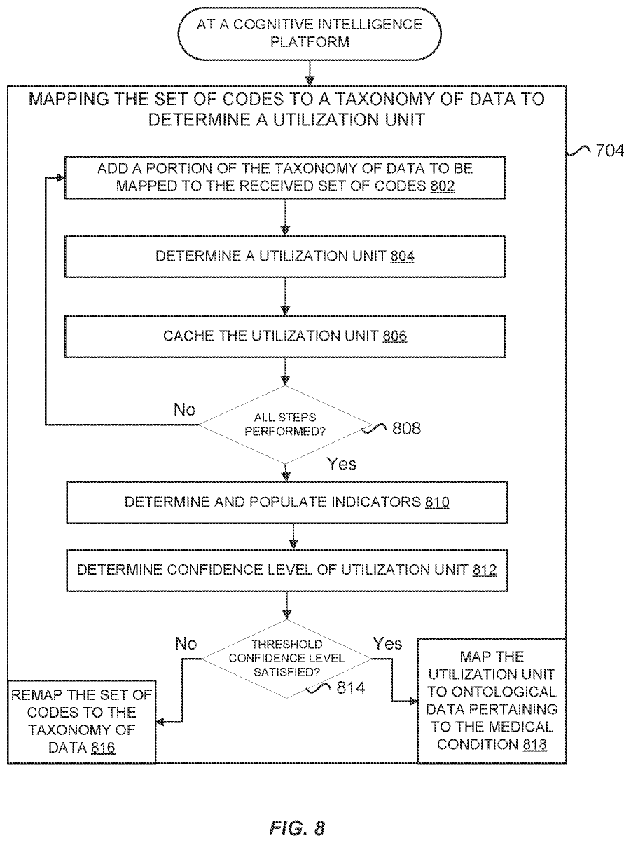

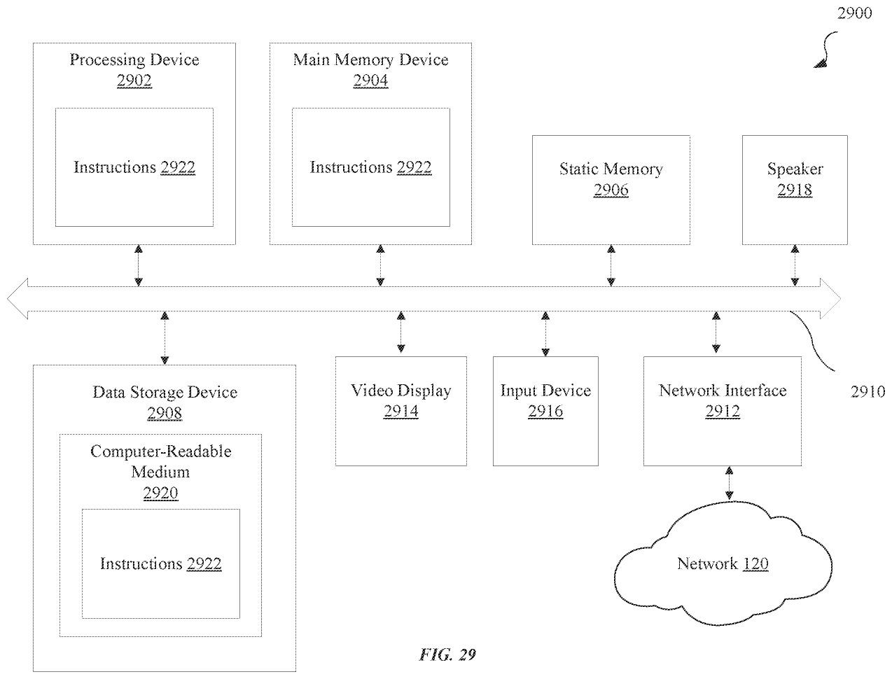

FIG. 7 shows an example method 700 for performing mapping operations to determine a knowledge fragment, in accordance with various embodiments. In some embodiments, the method 700 is implemented on a cognitive intelligence platform. In some embodiments, the cognitive intelligence platform is the cognitive intelligence platform 102 as shown in FIG. 1. In some embodiments, the cognitive intelligence platform is implemented by one or more of the computing devices 2900 shown in FIG. 29. The method 700 may include operations that are implemented in computer instructions stored in a memory and executed by a processor of a computing device. In some embodiments, the method 700 includes operations performed by the cognitive agent 110 (autonomous multipurpose application), the knowledge cloud 106, and/or the critical thinking engine 108 of the cognitive intelligence platform 102 as shown in FIG. 1.

At block 702, the processing device may receive a set of codes 600 pertaining to an event performed for a patient. For example, the codes 600 may pertain to a surgery performed at a hospital for the patient. It should be understood that the codes 600 may pertain to any event performed for a patient at any facility 114.

At block 704, the processing device may map the set of codes 600 to a taxonomy of data 602 to determine a utilization unit 604. The mapping the set of codes to the taxonomy of data to determine the utilization unit may further include performing a stepwise heuristic of adding different portions of the taxonomy of the data during the mapping until a confidence threshold is satisfied. The mapping the set of codes 600 to the taxonomy of data 602 may further include mapping a relationship between the plurality codes and the taxonomy of the data to obtain the utilization unit. The method 800 in FIG. 8 illustrates additional details regarding mapping the set of codes 600 to the taxonomy of data 602.

At block 706, the processing device may map the utilization unit 604 to ontological data 606 of a medical condition. The ontological data may be included in a knowledge graph pertaining to a medical condition, a procedure, or the like.

At block 708, the processing device may map the ontological data 606 to a knowledge fragment 608 pertaining to the medical condition and the patient. In some embodiments, the knowledge fragment is provided as input from the computing device associated of the medical personnel, from an electronic medical record system, from a repository of evidence-based guidelines, from a repository of clinical trial results, or some combination thereof.

In some embodiments, the ontological data may be mapped to a difference between the ontological data and a data structure (e.g., patient graph) pertaining to the patient and the medical condition. The processing device may determine the knowledge fragment based on the difference. In some embodiments, an intervention may be performed based on the knowledge fragment. The intervention may include transmitting an action instruction to a display of a computing device, transmitting an electronic message, transmitting a text message, making a telephone call, dispatching a person, or some combination thereof.

The knowledge fragment may be determined based on the knowledge graph of the condition and/or procedure, and/or the patient graph of the patient. In some embodiments, the knowledge fragment 608 may be based on information provided by a physician, an evidence-based guideline, results of clinical trials, documents approved by certified medical professionals, and so forth.

At block 710, the processing device may cause the knowledge fragment 608 to be presented on a computing device of a medical personnel. For example, the medical personnel may perform a follow-up appointment for the patient after a surgery for a medical condition. The patient graph for the patient and the medical condition may indicate the patient had the surgery. A knowledge graph for the medical condition may indicate that if a patient has the surgery, the patient can do certain self-care actions to recover faster. The knowledge fragment may include an action instruction for the medical personnel to instruct the patient to perform the self-care actions.

FIG. 8 shows an example method 800 for mapping a set of codes 600 to a taxonomy of data 602 to determine a utilization unit 604, in accordance with various embodiments. As depicted, the method 800 may be performed as part of the block 704 from the method 700 in FIG. 7. In some embodiments, the method 800 is implemented on a cognitive intelligence platform. In some embodiments, the cognitive intelligence platform is the cognitive intelligence platform 102 as shown in FIG. 1. In some embodiments, the cognitive intelligence platform is implemented by one or more of the computing devices 2900 shown in FIG. 29. The method 800 may include operations that are implemented in computer instructions stored in a memory and executed by a processor of a computing device. In some embodiments, the method 800 includes operations performed by the cognitive agent 110 (autonomous multipurpose application), the knowledge cloud 106, and/or the critical thinking engine 108 of the cognitive intelligence platform 102 as shown in FIG. 1.

The method 800 may implement a heuristic, such as fuzzy heuristic that uses a stepwise approach to determining a utilization unit. A fuzzy heuristic may refer to solving a problem based on aggregating fuzzy numbers and combined precedence constraints. Fuzzy heuristics may be useful when uncertainty is involved, such as when codes are missing and/or inaccurate when received from EMR systems, claims systems, provider (e.g., medical, insurance) systems, and the like. The heuristic may include one or more steps.

At block 802, a first step may be performed where the processing device may add a portion of the taxonomy of data 602 to be mapped to the received set of codes 600. For example, COS1 may be added to be mapped with the set of codes 600. The processing device may compare the set of codes to the codes in COS1 and determine which mappings are found. An example mapping may include “code 12345” represents “admit in-patient”.

At block 804, the processing device may determine a utilization unit based on the mappings identified. For example, if a mapping from a code to data indicates that the service performed was inexpensive, then the utilization unit is not CABG, which is very expensive. If the mapping indicates that there was a surgery performed, the surgery was in-patient, the surgery required more than 1 day stay at the hospital, the surgery was a coronary procedure, and so forth, then CABG may be determined to be the utilization unit 604.

At block 806, the processing device may cache the utilization unit 604 in a memory device.

At block 808, the processing device may determine whether all steps defined in a heuristic are performed. If so, the processing device may determine and populate indicators at block 810.

If not all the steps are performed, the processing device may return to block 802 and perform a next step by adding another portion (e.g., COS2) of the taxonomy of data 602 to the previous portion (e.g., COS1) added. The processing device may proceed to block 804 to determine a utilization unit 604. The processing device may cache the utilization unit 806. The processing device may determine whether all steps defined in the heuristic are performed. If not, the processing device may return to block 802 to continue executing blocks 802, 804, 806, and 808 until all the steps in the heuristic have been performed. When the steps are performed, the processing device may determine and populate indicators at block 810.

Determining and populating indicators may include analyzing the various mappings identified and setting the indicators to a certain value. The indicators may include a “SingleDay Flag”, “LowCost Flag”, and/or “Surgery Flag”. Any suitable indicators may be used by the disclosed techniques. The indicators may be set to 0 or 1, or any suitable value. The indicators values may be cached.

At block 812, the processing device may determine a confidence level of the determined utilization unit(s) 604. To determine the confidence level, a point may be assigned to each step. The point may be assigned based on the utilization unit determined at the step, the mappings identified at the step, or some combination thereof. Further, a weight may be applied to each of the indicators and the weighted indicators may be summed with the points in the steps. The confidence level may be determined based on total points and agreement between the points of the steps. The confidence level may High, Medium, or Low.

At block 814, the processing device may determine whether a threshold confidence level is satisfied (e.g., Medium or High). If so, the processing device may map (block 818) the utilization unit to ontological data pertaining to the medical condition. If the threshold confidence level is not satisfied (e.g., Low), the processing device may remap (block 816) the set of codes to the taxonomy of data by restarting the method 800.

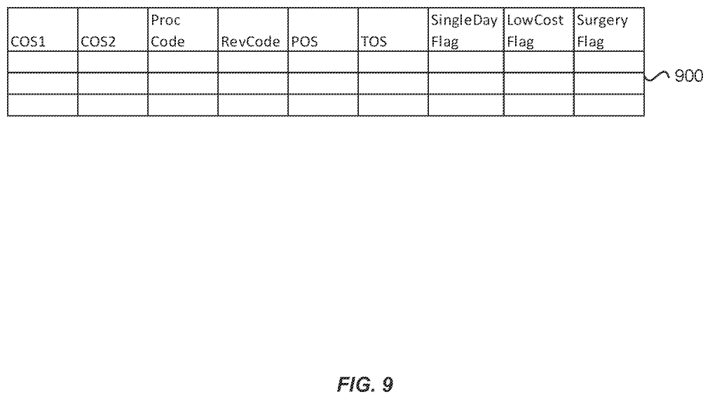

FIG. 9 shows an example table 900 used to cache data used or output by the method 800 of FIG. 8, in accordance with various embodiments. The table includes various code-related columns “COS1”, “COS2”, “Proc Code”, “RevCode”, “POS”, “TOS” and various indicator columns “SingleDay Flag”, LowCost Flag”, and “Surgery Flag”.

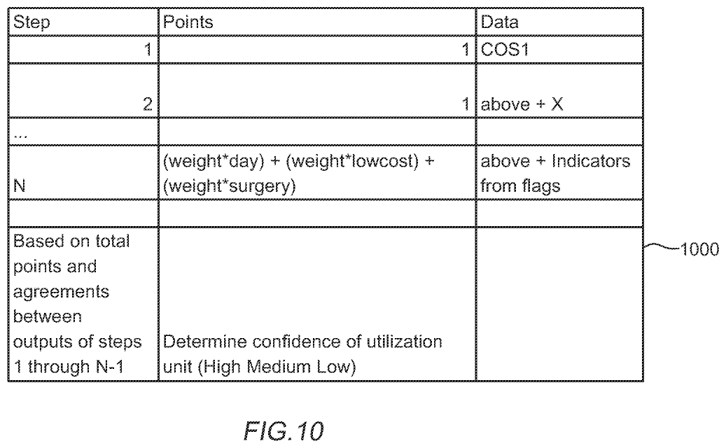

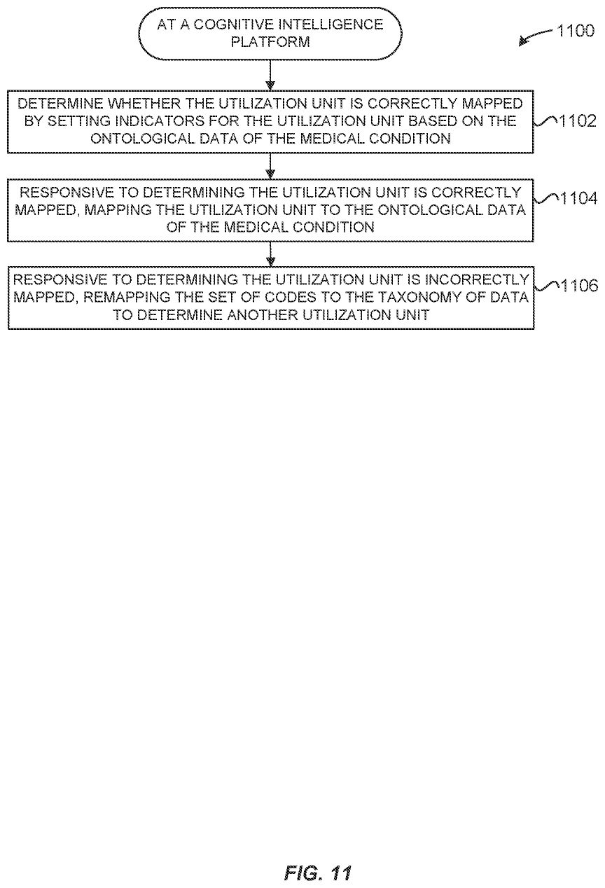

FIG. 10 shows an example table 1000 used to determine a confidence level of the determined utilization unit, in accordance with various embodiments. as depicted, the table 1000 includes a column for “Step”, “Points”, and “Data”. The Steps column includes rows for the number of steps and an Nth row for summing weighted indicators. As depicted, Step 1 added data from COS1 to be mapped against the codes 600. Step 2 used the first added data from COS1 and added “X”, where X may be any other data in the taxonomy, such as COS2, COS3, COSN, etc.

The total points may be determined by summing the values in the Points column for the steps and the weighted indicators. The last row indicates that the confidence value is based on the total points and agreement between outputs of steps 1 through N−1.

FIG. 11 shows an example method 1100 for determining whether a utilization unit 604 is correctly mapped, in accordance with various embodiments. In some embodiments, the method 1100 is implemented on a cognitive intelligence platform. In some embodiments, the cognitive intelligence platform is the cognitive intelligence platform 102 as shown in FIG. 1. In some embodiments, the cognitive intelligence platform is implemented by one or more of the computing devices 2900 shown in FIG. 29. The method 1100 may include operations that are implemented in computer instructions stored in a memory and executed by a processor of a computing device. In some embodiments, the method 1100 includes operations performed by the cognitive agent 110 (autonomous multipurpose application), the knowledge cloud 106, and/or the critical thinking engine 108 of the cognitive intelligence platform 102 as shown in FIG. 1.

At block 1102, the processing device may determine whether the utilization unit 604 is correctly mapped by setting indicators for the utilization unit based on the ontological data of the medical condition. For example, if the SingleDay Flag is set and the utilization unit determined is coronary artery bypass graft (CABG), then the processing device may determine that the utilization unit is not correctly mapped. If the indicators are appropriate for the utilization unit, then the utilization unit is determined to be correctly mapped.

At block 1104, responsive to determining the utilization unit is correctly mapped, the processing device may map the utilization unit to the ontological data of the medical condition. For example, if the utilization unit is CABG, then the ontological data for coronary artery disease may be mapped. In another example, if the utilization unit is CABG, then ontological data for the CABG procedure may be mapped. At block 1106, responsive to determining the utilization unit is incorrectly mapped, the processing device may remap the set of codes to the taxonomy of data to determine another utilization unit.

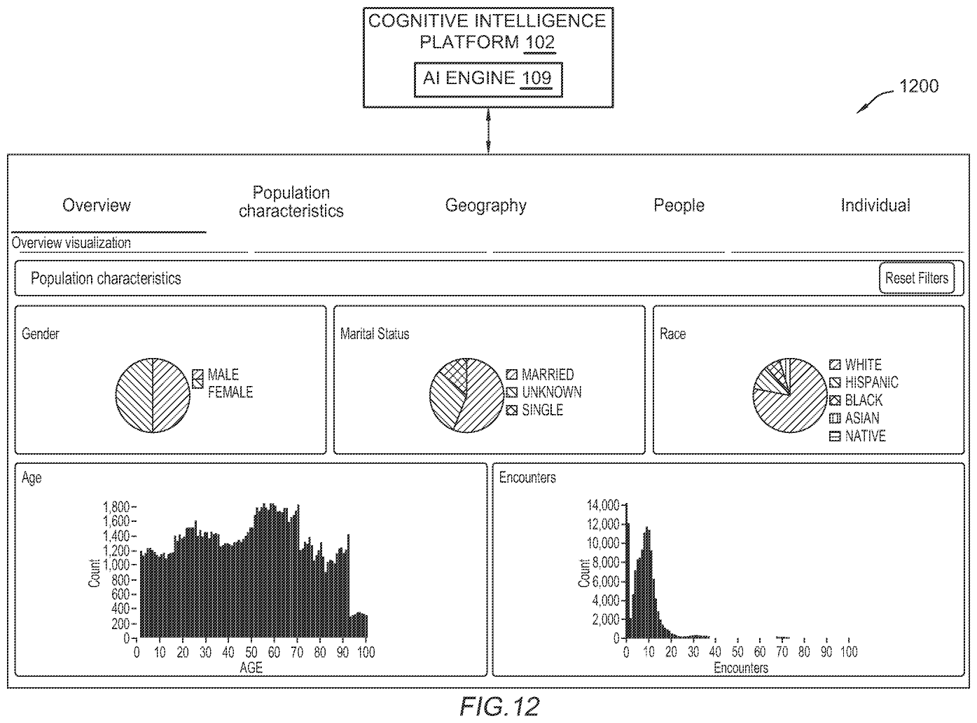

FIG. 12 shows an example user interface 1200 for population characteristics, in accordance with various embodiments. The user interface 1200 may be generated based on a set of data pertaining to patients in a population (e.g., city-wide, state-wide, country-wide, the world). The set of data may be stored in the cognitive intelligence platform 102 and may be obtained from any suitable source that provides information pertaining to patients (e.g., EMR systems, claims systems, third-party systems, etc.). The set of data may be used to generate profile graphs for each of the patients. A patient graph may be generated for each medical condition for each patient. The population profile may include the aggregated data depicted in the patient graphs to provide insights to how effective clinical programs are, to identify outliers (e.g., a physician is illegally prescribing opioids), etc.

The dynamic dashboards presented using the population profile may enable a medical personnel to view clinical data of a population at a high level and drill-down to any given individual patient to view information in a patient graph pertaining to that patient. The dynamic dashboards may update in real-time (e.g., less than 2 seconds) as information pertaining to the patients is received by the cognitive intelligence platform 102. The population profile provides a complete clinical orientation to each patient and how compliant each patient is across the entire population. The use of the population profile is a true management of risk that uses clinical informatics to change behaviors of patients.

As depicted, the user interface 1200 includes dashboards for “Gender”, “Marital Status”, “Race”, “Age”, and “Encounters”. An encounter may refer to a patient visiting a facility 114 to have a person render a service (e.g., a doctor's visit, surgery at a hospital, etc.). The population characteristics may be provided in the dashboards from the AI engine 109 of the cognitive intelligence platform 102.

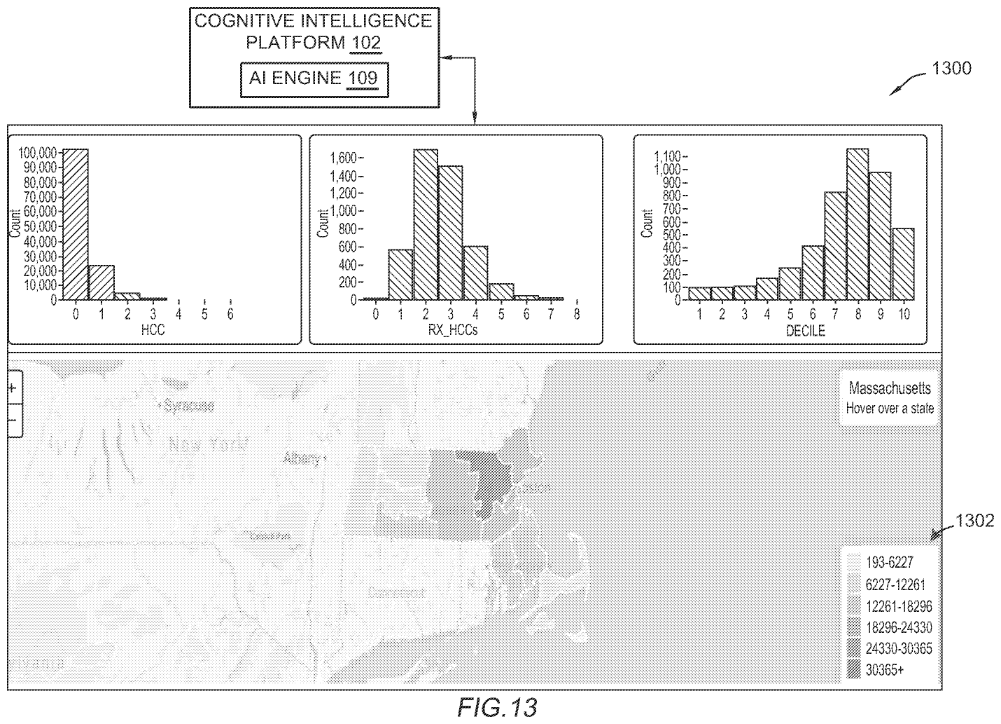

FIG. 13 shows an example user interface 1300 for managing risk associated with a medical condition at a population level, in accordance with various embodiments. The user interface 1300 includes dynamic dashboards for “HCC”, “RX_HCCs”, “Decile”, and a graphic of the country that enables selecting a particular state for which to view a population profile. In the depicted user interface 1300, the state of Massachusetts is selected. A legend 1302 is included in the graphic that shows colors correlated with certain ranges of people. For example, “yellow” may be correlated with 193-6227 people and “dark red” may be correlated with 30365+ people.

The medical condition presented in the user interface 1300 is Hepatocellular Carcinoma (HCC). The dashboard for HCC indicates that less than 10,000 patients have stage 2 HCC in Massachusetts. To view details pertaining to those patients, a user may select the bar representing the patients having stage 2 HCC in the HCC dashboard and another user interface may be presented for a patient having HCC. The user interface may present information that enables determining how compliant with a care plan the user has been in relation to the other patients having HCC in the population. Certain discrepancies and/or gaps in treatment for the patient may be determined based on non-compliance and certain action instructions may be provided by the AI engine 109 of the cognitive intelligence platform 102. The action instructions may include instructions for the patient and/or the user (medical personnel) to take actions to make the patient compliant with a care plan for HCC.

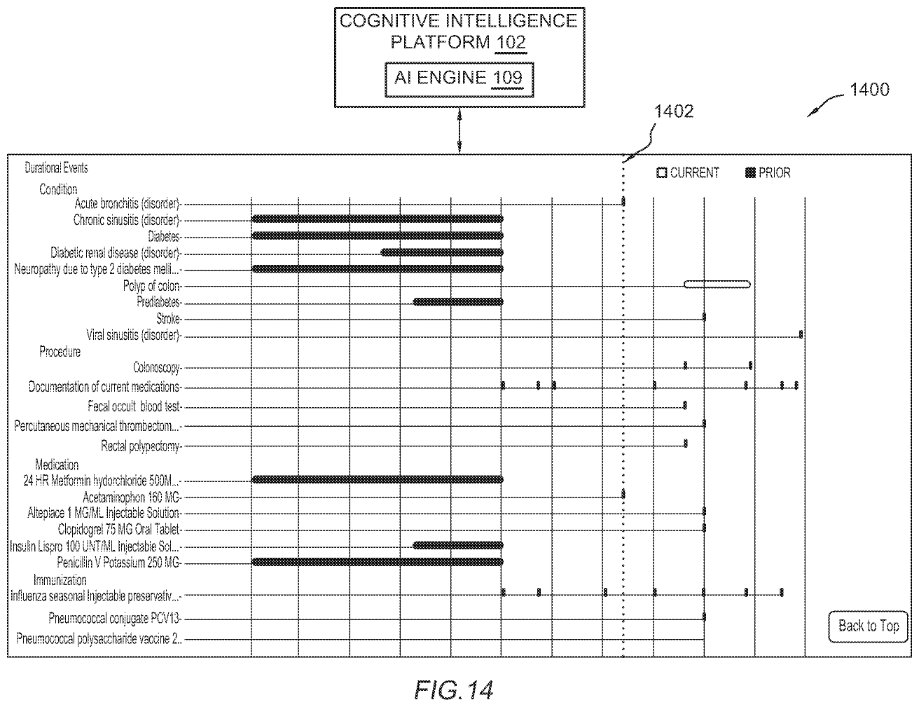

FIG. 14 shows an example user interface 1400 presenting durational events for a patient, in accordance with various embodiments. The user interface 1400 may be presented after a user has selected to drill-down from a user interface displaying information presented at the population level. The user interface 1400 depicts durational events for Conditions, Procedures, Medications, and Immunizations. The durational events are depicted as extending over a period of time on a timeline. The user interface 1400 includes current and prior data.

The various durational events in the user interface 1400 may relate to each other based on being vertically aligned in the user interface 1400. For example, line 1402 indicates that two durational events correspond to and relate to each other. The two durational events include “Acute bronchitis disorder” and “Acetaminophen 160 MG”. The user interface 1400 may also present event information episodically.

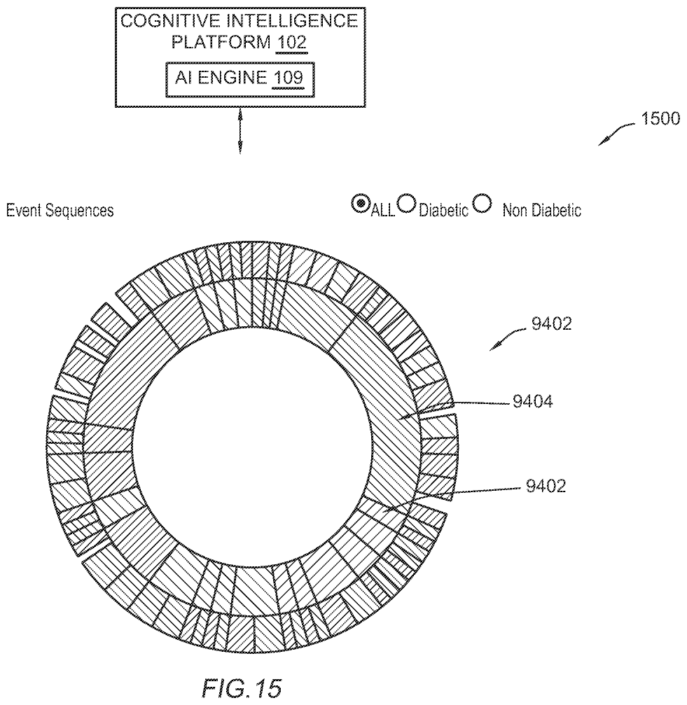

FIG. 15 shows an example user interface 1500 presenting a graphical element of event sequences for a patient over a certain time period, in accordance with various embodiments. The user interface 1500 may be presented after a user has selected to drill-down from a user interface displaying information presented at the population level. The user interface 1500 includes a ring graphic 1502 that presents information about the patient pertaining to event sequences over a period of time (e.g., a week, a month, a year, numerous years, a life of the patient, etc.).

Each portion of the ring may represent a different event (e.g., taking a medication, doctor visit, procedure performed, disease, condition, etc.) pertaining to healthcare. For example, a portion 1504 of the ring graphic 1502 may represent that a patient was taking a prescribed medication for 6 months. Another portion 1504 following portion 1502 may represent an event of the patient attending a follow-up appointment with a physician and discontinuing use of the medication. The user may use a cursor to hover over any portion of the ring graphic 1502 to view the details pertaining to the event represented at that portion. The user may view each of their healthcare related events quickly and easily using the ring graphic 1502. Accordingly, the ring graphic 1502 provides an enhanced graphical user interface that may improve a user experience using a computing device.

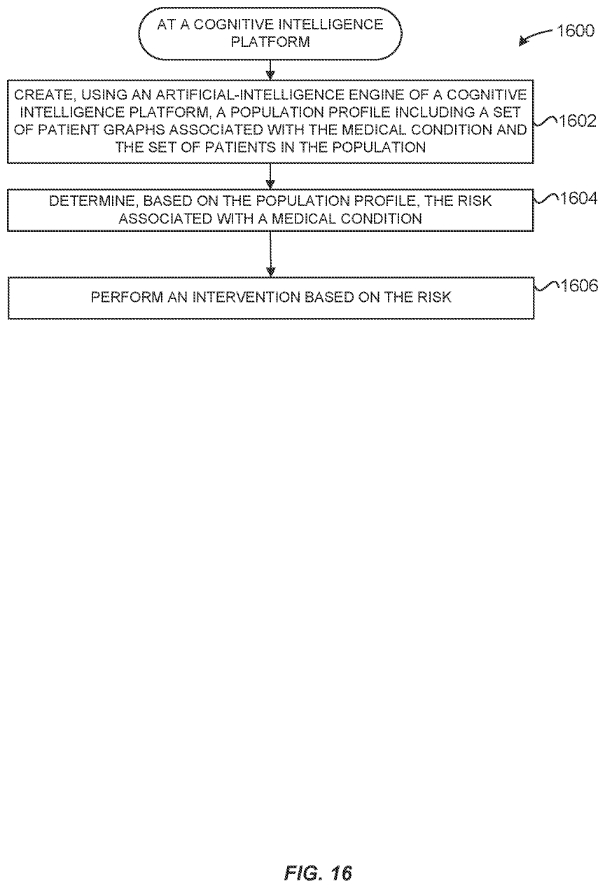

FIG. 16 shows an example method 1600 for using a population profile to perform an intervention, in accordance with various embodiments. In some embodiments, the method 1600 is implemented on a cognitive intelligence platform. In some embodiments, the risk includes a potential inadequacy in management of the medical condition. In some embodiments, the cognitive intelligence platform is the cognitive intelligence platform 102 as shown in FIG. 1. In some embodiments, the cognitive intelligence platform is implemented by one or more of the computing devices 2900 shown in FIG. 29. The method 1600 may include operations that are implemented in computer instructions stored in a memory and executed by a processor of a computing device. In some embodiments, the method 1600 includes operations performed by the cognitive agent 110 (autonomous multipurpose application), the knowledge cloud 106, and/or the critical thinking engine 108 of the cognitive intelligence platform 102 as shown in FIG. 1. The method 1600 may include operations to manage risk associated with a medical condition diagnosed for a set of patients in a population.

At block 1602, the processing device may create, using an artificial-intelligence engine of a cognitive intelligence platform 102, a population profile including a set of patient graphs associated with the medical condition and the set of patients in the population. In some embodiments, the processing device may cause the population profile or aspects of the population profile to be presented in a user interface of a computing device of a medical personnel, where the population profile is presented in or more graphical elements selected from a group of graphical elements including graphs, charts, and natural language.

In some embodiments, the processing device may receive a selection to drill-down into a patient graph in the set of patient graphs in the population profile. Responsive to receiving a selection to organize the information in the patient graph episodically, the processing device may organize the information in the patient graph episodically. Responsive to receiving a selection to organize the information in the patient graph by duration, the processing device may organize the information in the patient graph by duration. The processing device may cause the information in the patient graph to be presented in the user interface.

At block 1604, the processing device may determine, based on the population profile, the risk associated with a medical condition. In some embodiments, the processing device may determine, based on the patient graph of the set of patient graphs, a level of compliance of a patient in the set of patients in relation to other patient graphs in the set of patient graphs for other patients in the set of patients, and the processing device may determine the risk based on the level of compliance. In some embodiments, the level of compliance may relate to management of the medical condition. For example, the AI engine 109 of the cognitive intelligence platform 102 may track the medical conditions that occur or are diagnosed for each patient in the population profile. The AI engine 109 may also track the interactions the patients have with those medical conditions over time in the respective patient graphs. The patients that experience better medical results than other patients may be identified and the differences between the management of the medical condition by the patients may be identified based on the interactions stored in the patient graphs.

In one example, a first patient has performed a first set of interactions with health artifacts in a first patient graph for a first medical condition and the first patient is diagnosed with a second medical condition. A second patient has performed a second set of interactions with the health artifacts in a second patient graph for the same first medical condition. The second set of interactions may be less than the first set of interactions. The AI engine 109 may compare the second patient graph for the second patient and the first medical condition with the first patient graph for the first patient and the first medical condition. Based on the comparison, the AI engine 109 may determine the second patient is less compliant with a care plan for the first medical condition and is at risk for contracting the second medical condition.

If the level of compliance indicates the patient is below a threshold level (e.g., 30%) for managing the medical condition in relation to compliance levels of other patients in the population, then the processing device may determine the patient is at risk for an undesirable medical outcome. In some embodiments, the compliance level of managing the medical condition for each patient may be determined based on a percentage of completion of interactions and/or action instructions included in a care plan for each patient for the medical condition.

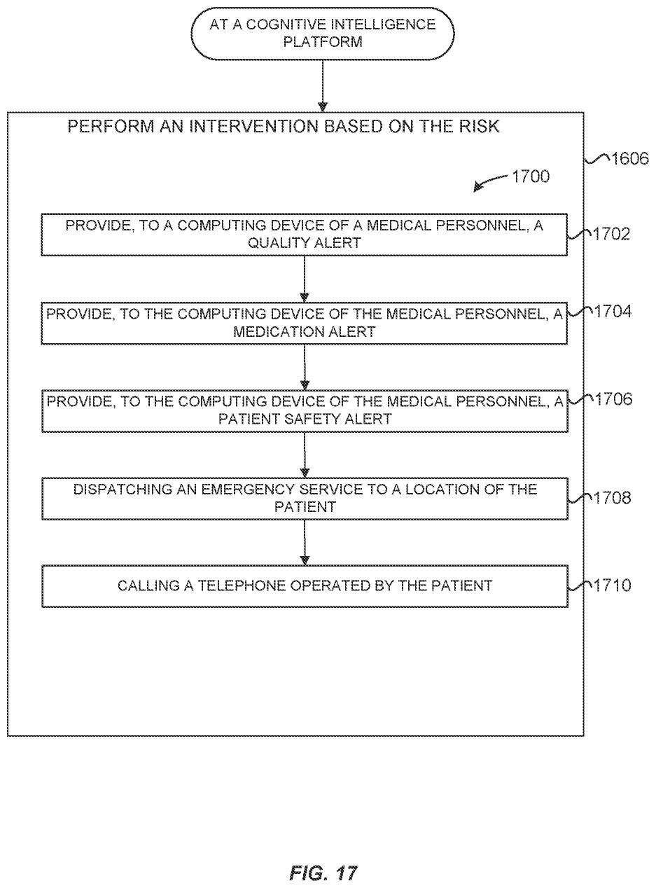

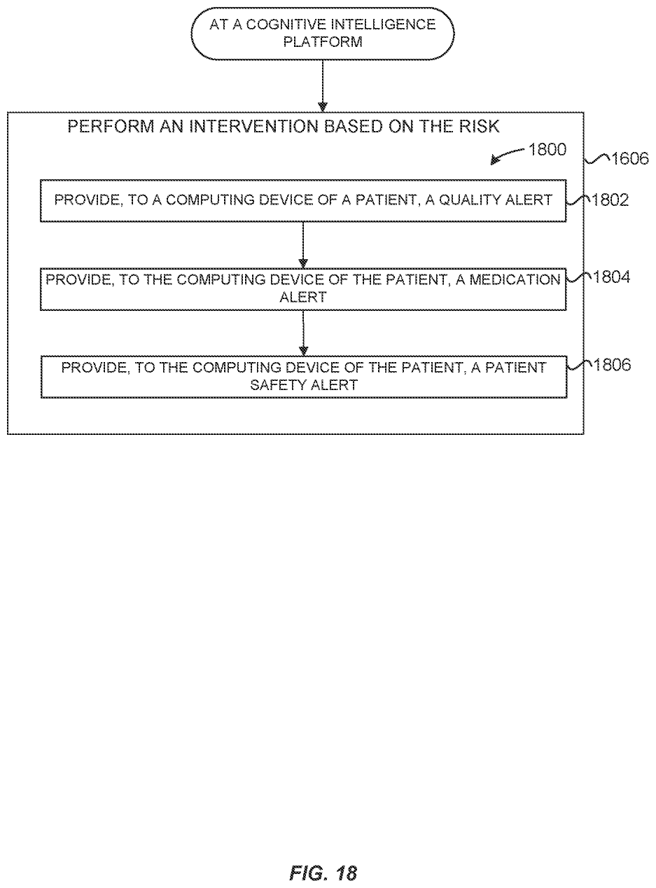

At block 1606, the processing device may perform an intervention based on the risk. The interventions are described further below with regard to the method 1700 in FIG. 17 and the method 1800 in FIG. 18.

In some embodiments, the processing device may segment the population profile into a set of segments including a respective subset of patient graphs of the set of patient graphs. The segmenting may be performed based on a compliance level with management of the medical condition, a type of medical condition diagnosed for the set of patients, a type of medicine prescribed to the set of patients, or some combination thereof.

FIG. 17 shows an example method 1700 for performing the intervention based on a risk, in accordance with various embodiments. As depicted, the method 1700 may be performed as part of the block 1606 of the method 1600 of FIG. 16. In some embodiments, the method 1700 is implemented on a cognitive intelligence platform. In some embodiments, the cognitive intelligence platform is the cognitive intelligence platform 102 as shown in FIG. 1. In some embodiments, the cognitive intelligence platform is implemented by one or more of the computing devices 2900 shown in FIG. 29. The method 1700 may include operations that are implemented in computer instructions stored in a memory and executed by a processor of a computing device. In some embodiments, the method 1700 includes operations performed by the cognitive agent 110 (autonomous multipurpose application), the knowledge cloud 106, and/or the critical thinking engine 108 of the cognitive intelligence platform 102 as shown in FIG. 1.

At block 1702, the intervention may include the processing device providing, to a computing device of a medical personnel, a quality alert. The quality alert may include an action instruction for the medical personnel to perform to comply with evidence-based guidelines pertaining to the medical condition.

At block 1704, the intervention may include the processing device providing, to the computing device of the medical personnel, a medication alert. The medication alert may include an action instruction for the medical personnel to perform pertaining to medication for the patient.

At block 1706, the intervention may include the processing device providing, to the computing device of the medical personnel, a patient safety alert. The patient safety alert may include an action instruction for the medical personnel to perform to safely render a service for the patient (e.g., the patient is allergic to latex, do not wear latex gloves).

At block 1708, the processing device may dispatch an emergency service to a location of the patient.

At block 1710, the processing device may call a telephone operated by the patient.

FIG. 18 shows another example method 1800 for performing the intervention based on the risk, in accordance with various embodiments. As depicted, the method 1800 may be performed as part of block 1606 of the method 1600 in FIG. 16. In some embodiments, the method 1800 is implemented on a cognitive intelligence platform. In some embodiments, the cognitive intelligence platform is the cognitive intelligence platform 102 as shown in FIG. 1. In some embodiments, the cognitive intelligence platform is implemented by one or more of the computing devices 2900 shown in FIG. 29. The method 1800 may include operations that are implemented in computer instructions stored in a memory and executed by a processor of a computing device. In some embodiments, the method 1800 includes operations performed by the cognitive agent 110 (autonomous multipurpose application), the knowledge cloud 106, and/or the critical thinking engine 108 of the cognitive intelligence platform 102 as shown in FIG. 1.

At block 1802, the intervention may include the processing device providing, to a computing device of a patient, a quality alert. The quality alert may include an action instruction for the patient to perform to comply with evidence-based guidelines pertaining to the medical condition.

At block 1804, the intervention may include the processing device providing, to the computing device of the patient, a medication alert. The medication alert may include an action instruction for the patient to perform pertaining to medication for the patient.

At block 1806, the intervention may include the processing device providing, to the computing device of the patient, a patient safety alert. The patient safety alert may include an action instruction for the patient to perform to safely render a service for the patient (e.g., the patient is allergic to latex, do not wear latex gloves).

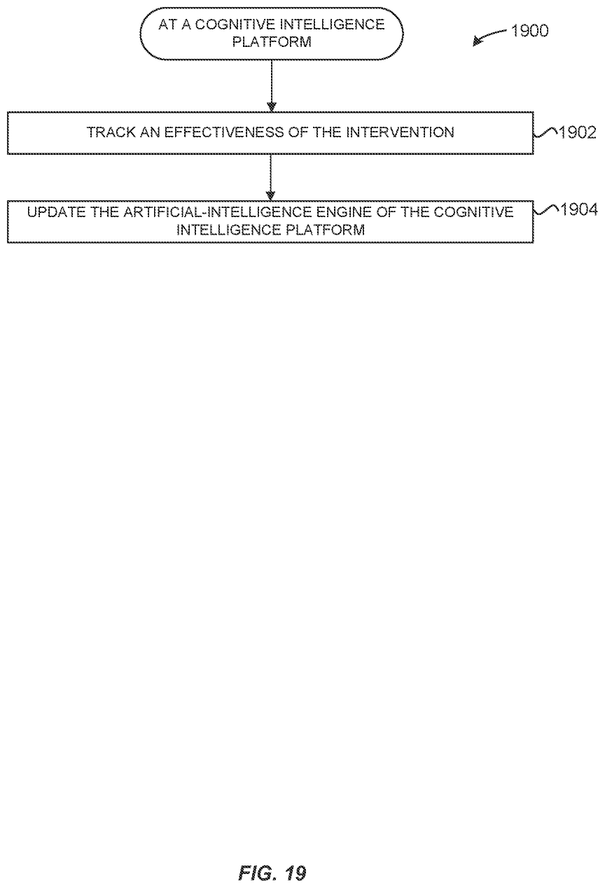

FIG. 19 shows an example method 1900 for updating an artificial-intelligence engine based on an effectiveness of an intervention, in accordance with various embodiments. In some embodiments, the method 1900 is implemented on a cognitive intelligence platform. In some embodiments, the cognitive intelligence platform is the cognitive intelligence platform 102 as shown in FIG. 1. In some embodiments, the cognitive intelligence platform is implemented by one or more of the computing devices 2900 shown in FIG. 29. The method 1900 may include operations that are implemented in computer instructions stored in a memory and executed by a processor of a computing device. In some embodiments, the method 1900 includes operations performed by the cognitive agent 110 (autonomous multipurpose application), the knowledge cloud 106, and/or the critical thinking engine 108 of the cognitive intelligence platform 102 as shown in FIG. 1.

At block 1902, the processing device may track an effectiveness of the intervention. The effectiveness may be tracked based on whether the medical condition improves or gets worse as a result of the intervention. Such a determination may be made upon receiving event information subsequent to the intervention being performed. For example, if the intervention specified the user takes a certain medication and the next event information is from a care provider indicating the medical condition is gone, then the processing device may determine the intervention improved the medical condition.

At block 1904, the processing device may update the artificial-intelligence engine of the cognitive intelligence platform 102. If the processing device determines that the intervention worsened the medical condition, then the processing device may update the AI engine 109 to perform a different intervention in the future.

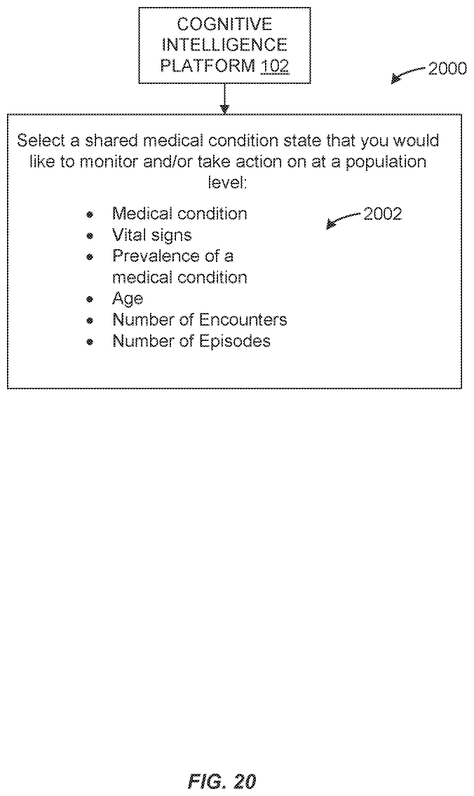

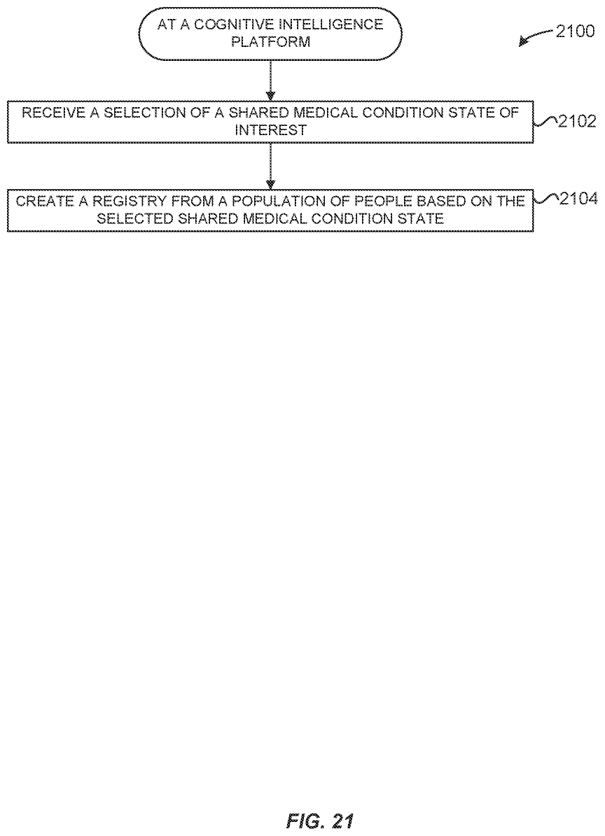

FIG. 20 shows an example user interface for selecting a shared medical condition state of interest to be monitored and/or acted upon at a population level, in accordance with various embodiments. FIG. 21 shows an example method 2100 for selecting a shared medical condition state of interest to be monitored and/or acted upon at a population level, in accordance with various embodiments. For purposes of clarity, FIGS. 20 and 21 are discussed together below.

In some embodiments, the method 2100 is implemented on a cognitive intelligence platform. In some embodiments, the cognitive intelligence platform is the cognitive intelligence platform 102 as shown in FIG. 1. In some embodiments, the components (e.g., the knowledge cloud 106, the critical thinking engine 108, the AI engine 109, the cognitive agent 110, and/or the natural language database 122) of the cognitive intelligence platform may be implemented by one or more of the computing devices 2900 of FIG. 29. The method 2100 may include operations that are implemented in computer instructions stored in a memory and executed by a processor of a computing device. In some embodiments, the method 2100 includes operations performed by the cognitive agent 110 (autonomous multipurpose application), the knowledge cloud 106, and/or the critical thinking engine 108 of the cognitive intelligence platform 102 as shown in FIG. 1.

At block 2102, the processing device may receive a selection of a shared medical condition state of interest at a population level. The selection may be received from the computing device of a user (e.g., medical personnel) via a graphical element 2002 of a user interface 2000 of FIG. 20. For example, the graphical element 2002 may include text stating “Select a shared medical condition state that you would like to monitor and/or take action on at a population level”. The shared medical condition state may also refer to a criteria and may pertain to people, actions, and/or things.

For example, regarding people, the shared medical condition state, as depicted, may include a medical condition, vital signs, a prevalence of a medical condition, an age of people, a number of encounters (e.g., events such as appointments, medications, procedure being performed, etc.), a number of episodes (e.g., cluster of encounters) within a time period, and so forth. Regarding actions, the shared medical condition state may include procedures performed for a medical condition, vaccines administered, office visits for a medical condition, prescriptions written for a certain medication, and so forth. Regarding things, the shared medical condition state may refer to over the counter medication bought that is associated with certain medications, first aid supplies bought within a region within a time period, online search queries pertaining to a certain medical condition, and so forth. For example, an increased number of online search queries related to the flue originating from IP addresses within a similar geographic region within a similar time period may indicate that there is a flu outbreak in that area. Such information may be helpful for a healthcare organization in determining where to distribute additional flu vaccines.

Selecting a shared medical condition state to monitor may enable healthcare organizations and/or medical personnel to monitor various aspects related to medical conditions, effectiveness of treatments, and/or trends of medical conditions at a population level. In the depicted example, a medical personnel may have selected to monitor a particular medical condition (e.g., Diabetes) in a population of people in a certain geographic region (e.g., a city, state, country, etc.).

At block 2104, the processing device may create a registry of people from a population of people based on the selected shared medical condition state. A registry may refer to a list of people, actions, or things that are associated with the shared medical condition state selected to be monitored and/or acted upon. For example, if the shared medical condition state of interest selected to be monitored is the medical condition Diabetes in a geographic region, then the registry may include a list of people (e.g., subset of people) chosen from a population of people where the people in the list have Diabetes. The registry may exclude people in the population that do not have Diabetes. If the shared medical condition state of interest to be monitored is the procedure of CABG being performed, then the registry may include a list of instances of the CABG procedure being performed and the instances may include information about each procedure, such as the person on which the procedure was performed, a date and time the procedure was performed, an outcome of the procedure, and the like.

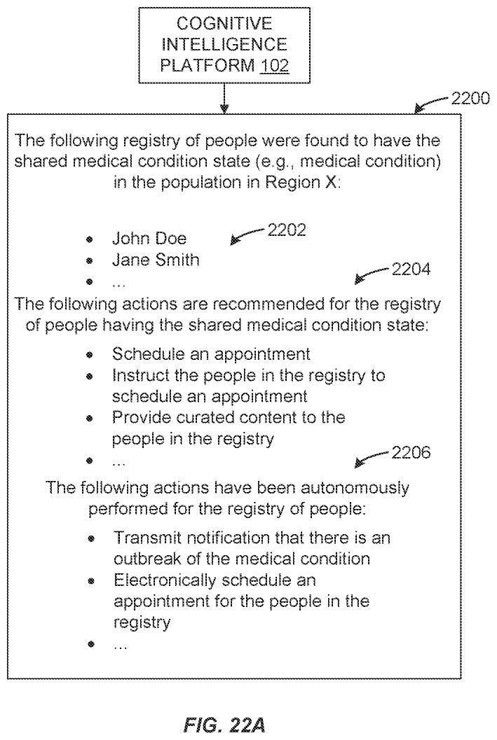

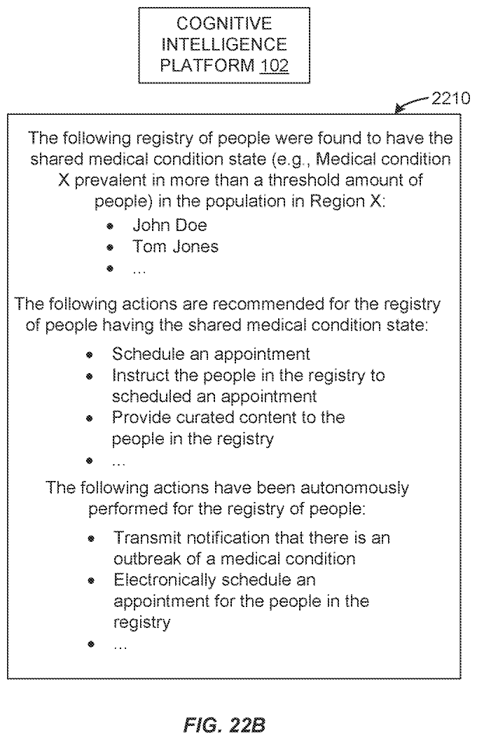

FIG. 22A shows an example user interface 2200 presenting a registry of people having a first shared medical condition state, recommended actions, and autonomously performed actions, in accordance with various embodiments. Although the depicted example presents a registry of people, it should be understood that any suitable type of registry of interest may be created to effectively track healthcare statistics and information at a population level. For example, the registry may include a list of actions and/or a list of things, as discussed further herein.

As discussed above, a shared medical condition state may have been previously selected by a medical personnel. In some embodiments, certain medical condition states may be selected by the cognitive intelligence platform 102 by default. The selections by the cognitive intelligence platform 102 may be based on learned trends in medical conditions peaking during certain seasons, in creating geographic regions, in certain genders and/or ethnicities, or the like. To that end, the cognitive intelligence platform 102 may use one or more machine learning models to select the shared medical condition state based on training data including preferences of medical conditions to track, procedures to track, and/or any suitable healthcare information of interest. In some embodiments, the medical personnel and the cognitive intelligence platform 102 may select a combination of shared medical condition states (e.g., the medical personnel may select some and the cognitive intelligence platform 102 may select some).

Once the shared medical condition state is selected, the cognitive intelligence platform 102 may create a registry of data having the shared medical condition state. The registry may include a subset of data (e.g., people, actions, things) from a superset of data. In the depicted example, the registry includes a sub-population of people having a shared medical condition state (e.g., medical condition) from a population of people. The population of people may be configured to include people living within a certain geographic radius, having a certain race, a certain gender, a certain age range, and/or any suitable characteristic that qualifies a group of people into a population. The shared medical condition state may provide further qualification to segment the population of people into a registry of people 2202. The registry of people 2202 may include numerous information about the people in the registry, such as the names of the people, the ages, the medical conditions, the medical history, the genders, the ethnicities, residency, and so forth. As depicted, the user interface 220 presented on the computing device of the medical personnel indicates that “The following registry of people were found to have the shared medical condition state (e.g., medical condition) in the population in Region X: John Doe, Jane Smith, . . . ”. Accordingly, information about the registry of people is presented in the user interface 2200 to the medical personnel.

The cognitive intelligence platform 102 may consult a knowledge graph (e.g., taxonomy of ontological medical knowledge) associated with the shared medical condition state. For example, if the shared medical condition state of interest is Diabetes, then the knowledge graph corresponding to Diabetes may be referenced to determine an action to recommend and/or autonomously perform for the registry of people 2202. The action may be selected based on any health artifact and relationship included in the knowledge graph for Diabetes. For example, if the registry of people have just been diagnosed with Diabetes, the cognitive intelligence platform 102 may compare the new diagnosis to a root node of the knowledge graph for Diabetes and determine that Diabetes is treated by certain medications. The action may include prescribing the medication to the registry of people having Diabetes. In some embodiments, individual patient graphs for each person in the registry may be compared to the knowledge graph for Diabetes and a gap between the respective patient graph and the knowledge graph may be identified. The recommended action for each person in the registry may be individually tailored to that person. For example, a first person in the registry may have already been tested with a first test but not a second test, while a second person has not been tested by either test as determined by comparing the respective patient graphs to the knowledge graph of Diabetes. The action may include recommending performing the second test for the first person and performing either the first or the second test for the second person. In the depicted user interface 2200, a recommended action portion 2204 presents “The following actions are recommended for the registry of people having the shared medical condition state:
Schedule an appointment
Instruct the people in the registry to schedule an appointment
Provide curated content to the people in the registry
. . . ”

The user interface 2200 also includes a portion 2206 of actions that have been autonomously performed by the cognitive intelligence platform 102. These actions may be electronically performed once the registry of people 2202 is created and the action is determined based on referencing the knowledge graph associated with the shared medical condition state and/or the respective patient graphs of each person in the registry. The user interface 2206 presents “The following actions have been autonomously performed for the registry of people:
Transmit notification that there is an outbreak of the medical condition
Electronically schedule an appointment for the people in the registry
. . . ”

In some embodiments, it should be noted that any of the recommended actions may be autonomously performed and any of the autonomous actions may be recommended to the medical personnel to perform.

FIG. 22B shows an example user interface 2210 presenting a registry of people having a second shared medical condition state, recommended actions, and autonomously performed actions, in accordance with various embodiments. In this example, the second shared medical condition state includes identifying a geographic region having a prevalence of people diagnosed with a medical condition X more than a threshold amount of people. The threshold of people may be any suitable amount. If threshold amount of people diagnosed with medical condition X is satisfied, a registry of the people in the geographic region may be created. The registry of people may include similar information as described above with reference to FIG. 22A. As should be appreciated, any suitable number of different registries of data may be created for monitoring. Further, any suitable actions may be recommended and/or autonomously performed for each of the different registries. In some embodiments, the actions performed for the registries may be different and/or the same.

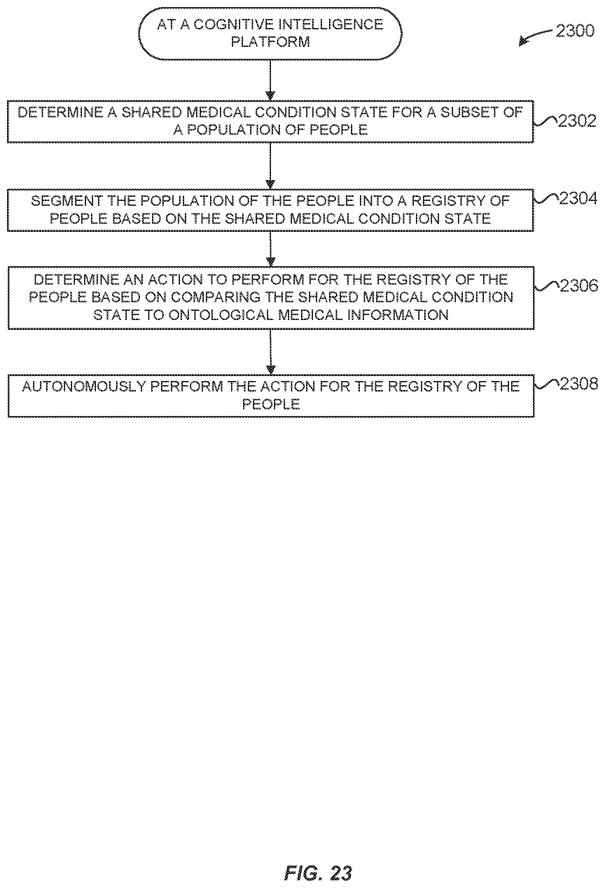

FIG. 23 shows an example method 2300 for segmenting a population into a registry and performing an action for the registry, in accordance with various embodiments. In some embodiments, the method 2300 is implemented on a cognitive intelligence platform. In some embodiments, the cognitive intelligence platform is the cognitive intelligence platform 102 as shown in FIG. 1. In some embodiments, the components (e.g., the knowledge cloud 106, the critical thinking engine 108, the AI engine 109, the cognitive agent 110, and/or the natural language database 122) of the cognitive intelligence platform may be implemented by one or more of the computing devices 2900 of FIG. 29. The method 2300 may include operations that are implemented in computer instructions stored in a memory and executed by a processor of a computing device. In some embodiments, the method 2300 includes operations performed by the cognitive agent 110 (autonomous multipurpose application), the knowledge cloud 106, and/or the critical thinking engine 108 of the cognitive intelligence platform 102 as shown in FIG. 1.

At block 2302, the processing device may determine a shared medical condition state for a subset of a population of people. The shared medical condition state may have been previously selected as described above with reference to FIGS. 20 and 21. The processing device may be communicatively connected to various EMR systems used by healthcare providers, to claims systems used by insurance providers, to appointment scheduling systems used by healthcare providers, and/or to systems (e.g., prescription management, transactional, etc.) used by pharmacies, convenience stores, groceries stores, etc. Accordingly, the processing device may receive information pertaining to medical condition states, such as when people are diagnosed with medical conditions, when appointments are scheduled for medical conditions, when medications are prescribed, when medications are purchased, when procedures are performed, and/or any type of healthcare related episode or interaction people experience.

At block 2304, the processing device may segment the population of the people into a registry of people based on the shared medical condition state. In some embodiments, the shared medical condition state may include a type of medical condition, a severity of the medical condition, a stage of the medical condition, encounters previously engaged by the people to treat the medical condition, age of the people, a prevalence of the medical condition, a number of the encounters, or some combination thereof.

For example, if the shared medical condition state of interest is a medical condition, such as Diabetes, the processing device may segment the population of the people into the registry based on which of the people are diagnosed with Diabetes or are determined to have a likelihood of having Diabetes based on any Diabetes related encounters or interactions. A Diabetes related encounter or interaction may include purchasing one or more at-home blood glucose tests within a certain time period and/or the results of the blood glucose tests indicating Diabetes, for example. The registry of people may include a subset of the people from the population of the people, where the subset of people have the shared medical condition state.

At block 2306, the processing device may determine an action to perform for the registry of the people based on comparing the shared medical condition state to ontological medical information. In some embodiments, the ontological medical information may be included in the knowledge graph 500 and may represent health artifacts that are connected via relationships. The ontological medical information may be based on evidence-based guidelines, clinical trials, medical personnel research, and the like.

In some embodiments, if the shared medical condition state of interest is the medical condition Diabetes and the registry of people includes a list of identities of people recently diagnosed with Diabetes, then the processing device my compare the recent diagnosis of Diabetes with ontological medical information pertaining to Diabetes. The processing device may determine, based on the ontological medical information for Diabetes, to provide certain curated medical content describing self-care treatments for Diabetes to computing devices of people in the registry.

In some embodiments, the action may include electronically scheduling an appointment for a person in the registry of the people. The processing device may communicate with a third-party system, such as an appointment scheduling system of a healthcare provider to obtain the schedule of medical personnel having a certain specialty and to compare the schedule with a schedule of the person (e.g., the schedule of the person may be shared with or provided to the processing device) to find a time that is available to both the medical personnel and the person. Further, the processing device may schedule the available time and provide notifications to computing devise of the person and the medical personnel or the facility at which the medical personnel works.

In some embodiments, the action may include sending an alert to the user computing device 104 of the person. For example, if the shared medical condition state pertains to Diabetes, then the alert may include a recommendation for the person to schedule a follow-up appointment with their medical personnel to check the status of the Diabetes, or the alert may include links to medical content pertaining to Diabetes.

In some embodiments, the action may include calling the user device 104 of the person in the registry. For example, if the shared medical condition state is a missed doctor appointment for a severe medical condition the person has, then the processing device may call the smartphone of the person to remind the person to reschedule the appointment and/or check on the status of the person.

In some embodiments, the action may include causing information to be presented via an audio device of the user device 104. For example, the information may pertain to scheduling an appointment, recommending medical content, buying medication, seeking immediate medical service, and the like.

In some embodiments, the action may include causing information to be presented via a user interface and/or an audio device of the computing device of the medical personnel. For example, the information may include detailed information about the registries created (e.g., identities of people, encounters, events, actions, medications, geographic region, timelines, etc.) and allow the medical personnel to drill down into the registries from a list level to an individual person detail level. To that end, the processing device may present the actual medical codes entered for each person's healthcare encounter or interaction.

In some embodiments, the processing device may cause the shared medical condition state, in conjunction with a recommendation pertaining to the action, to be presented on a computing device of the medical personnel. For example, if the shared medical condition state pertains to a failure to purchase prescribed medication or to refill the prescribed medication, the shared medical condition state for the people of the registry may be presented on the computing device of the medical personnel and a recommendation may include contacting the registry of people to instruct them to pick up the prescribed medication.

At block 2308, the processing device may autonomously perform the action for the registry of the people. Autonomously performing the action may refer to the processing device electronically performing the action without the aid of a person. Accordingly, the cognitive intelligence platform 102 may generate registries of people having shared medical condition states, determine actions to perform for the registries, and autonomously perform the actions.

In some embodiments, the processing device may segment the population of the people into a second registry of the people based on a second shared medical condition state, as shown in FIG. 22B. The processing device may determine a second action to perform based on comparing the second shared medical condition state to the ontological medical information. The second action may be different than the action performed for the registry of people. Further, the processing device may perform the second action. Accordingly, any suitable number of shared medical condition states may be specified to be monitored and/or acted upon and any suitable number of registries may be created. The processing device may perform the same and/or different actions for each registry.

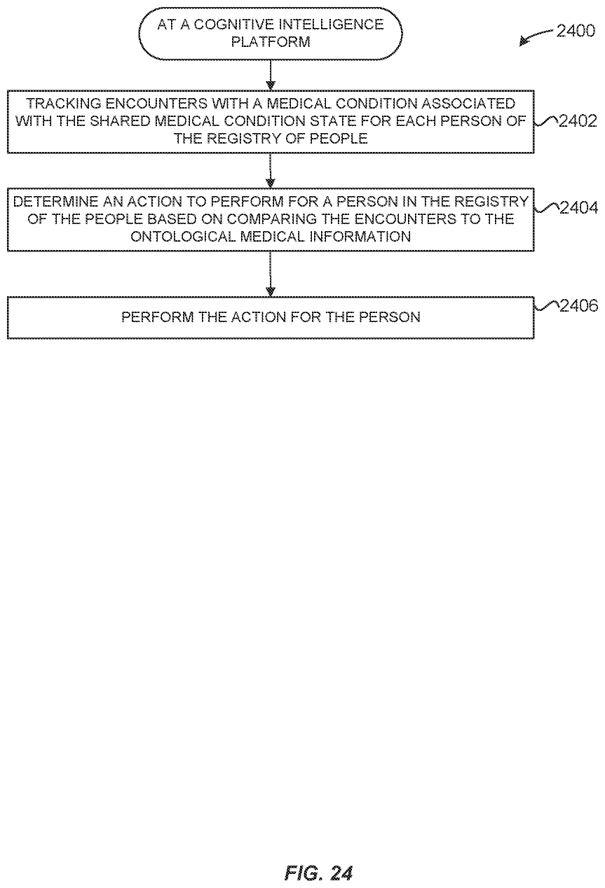

FIG. 24 shows an example method 2400 for tracking encounters with a medical condition associated with a shared medical condition state and performing an action based on the encounters, in accordance with various embodiments. In some embodiments, the method 2400 is implemented on a cognitive intelligence platform. In some embodiments, the cognitive intelligence platform is the cognitive intelligence platform 102 as shown in FIG. 1. In some embodiments, the components (e.g., the knowledge cloud 106, the critical thinking engine 108, the AI engine 109, the cognitive agent 110, and/or the natural language database 122) of the cognitive intelligence platform may be implemented by one or more of the computing devices 2900 of FIG. 29. The method 2400 may include operations that are implemented in computer instructions stored in a memory and executed by a processor of a computing device. In some embodiments, the method 2400 includes operations performed by the cognitive agent 110 (autonomous multipurpose application), the knowledge cloud 106, and/or the critical thinking engine 108 of the cognitive intelligence platform 102 as shown in FIG. 1.

At block 2402, the processing device may track encounters with a medical condition associated with the shared medical condition state for each person of the registry of people. Tracking the encounters may include receiving and/or requesting updates from EMR systems, claims systems, transactional systems, and so forth on a continuous or periodic basis. The encounters may include events such as visits to doctors, procedures being performed, and/or medication being purchased.

At block 2404, the processing device may determine an action to perform for a person in the registry of the people based on comparing the encounters to the ontological medical information. At block 2406, the processing device may perform the action for the person.

For example, when the shared medical condition state pertains to repeated doctor visits for Diabetes, the processing device may identify when a person having Diabetes visits the doctor for Diabetes (e.g., encounters) more than a threshold number of times within a threshold time period. The processing device may determine an action to perform for the person in the registry of the people based on comparing the encounters to the ontological medical information. The ontological medical information may indicate that visiting the doctor for Diabetes more than the threshold number of times within the threshold time period indicates the medication and/or treatment is not working. The processing device may determine the action is to recommend changing the medication and/or treatment. The processing device may perform the action by recommending to the medical personnel to change the medication and/or treatment.

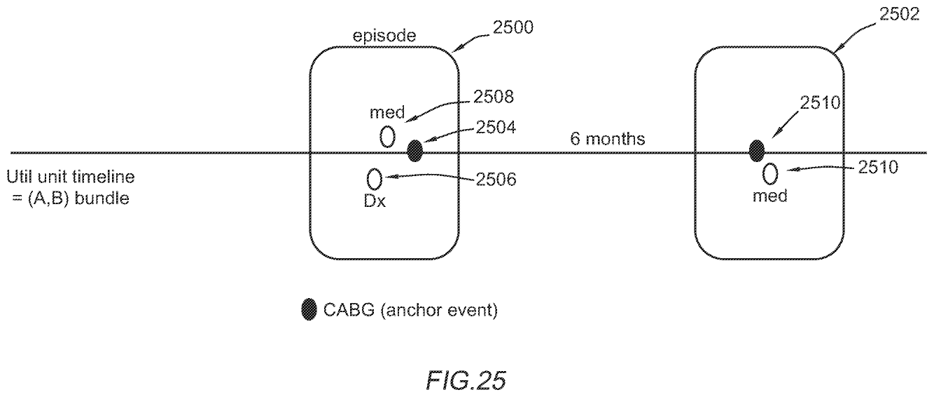

FIG. 25 shows an example of determining an anchor event 2508 and creating an episode 2500 of clustered events 2508 and 2506 around the anchor event 2504, in accordance with various embodiments. The episode 2500 may be referred to as a “clustered event episode bundle”. The example presents a mechanism for clustering durational events. In some embodiments, the cognitive intelligence platform 102 may create registries that are cross-referenced with the taxonomy of data (e.g., knowledge graph) to create clustered event episode bundles. An event for a person may include any health related interaction the person has, such as encounters with visiting the doctor, a procedure being performed, medication being prescribed, medication being bought, online medical queries via a website, and so forth. The events may be categorized into utilization units using the medical codes as described herein. The timestamps of the events may enable clustering the events relative in time to one another into bundles. A bundle may refer to a set of utilization categories in a medical condition. The episodes may be spaced apart on a timeline by days and weeks. The utilization unit timeline may be represented as “util unit timeline=(A,B) bundle”. The episode 2500 includes the events 2506 and 2508 and anchor event 2504 because they are relative in time to one another and are related to one another based on the taxonomy of medical knowledge associated with the anchor event 2504 (e.g., CABG). It should be noted that if there are other events that occur relative in time to the events 2506 and 2508 but they are not included in the taxonomy of knowledge for CABG, then those events may not be included in the episode 2500.

In the depicted example, the first episode includes the event 2506 occurring first for diagnosing the person with a medical condition and the event 2508 of a medication being prescribed. These events 2506 and 2508 may be determined based on the mapping of codes described herein. The events 2506 and 2508 may be placed on the utilization unit timeline. In some embodiments, the anchor event 2504 may be determined as the main healthcare related event that the other events in the episode relate to, depend on, and/or are associated with. In the depicted clustered event episode bundle 2500, The anchor event 2504 is Coronary Artery Bypass Graft (CABG).

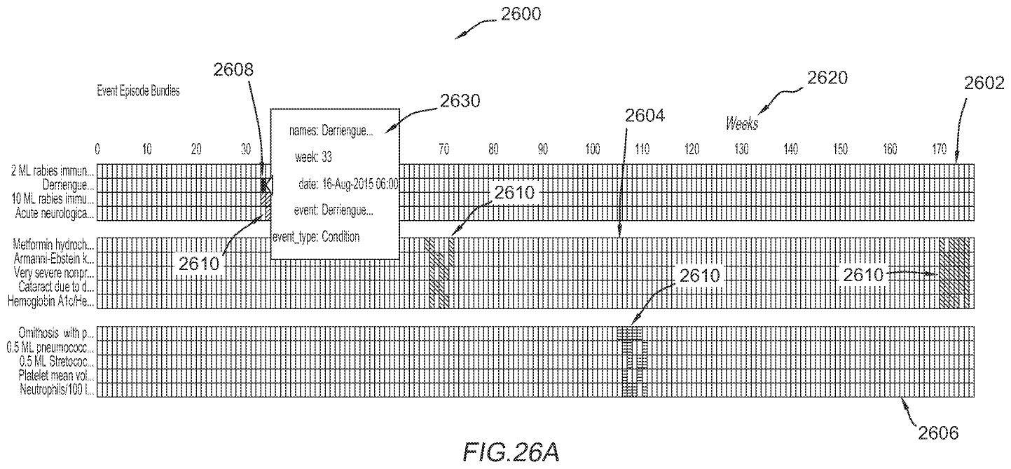

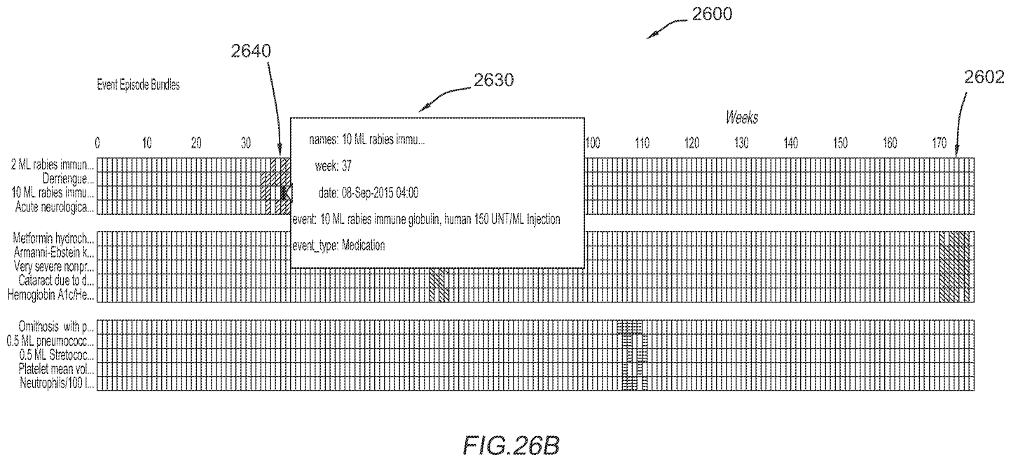

FIG. 26A shows an example user interface 2600 presenting clustered event episode bundles 2610 for various users on a timeline 2620, and autonomously performed actions, in accordance with various embodiments. The timeline 2620 is measured in weeks starting at week 0 and extending to week 180. Each square (box or object) in the clustered event episode bundles 2610 represent a respective event that is correlated to the particular medical condition as determined based on the taxonomy of medical knowledge and the time at which the event occurred. That is the events for the particular medical condition may be clustered into the clustered event episode bundles 2610. Each band 2602, 2604, and 2606 may represent certain medical conditions for the same person or different people.

A bundle may refer to a set of utilization categories for a medical condition. For example the band 2602 includes four rows representing utilization categories (2 ML rabis immune . . . , Derriengue . . . , 10 ML rabies immu . . . , Acute neuologica . . . ). Each square in each respective row represents an event occurring for that utilization category at that particular time on the timeline 2620. The user (medical personnel) may select one of the squares in the clustered event episode bundles to view detailed information about that event.

In some embodiments, the detailed information for the event may include a name, week, date, event, and an event type. As depicted, the user may select a first square 2608 for a first event in a row of the band 2602. The selection may cause a graphical element 2630 to be presented on the user interface 2600 and the graphical element 2630 may overlay or occlude a portion of one or more of the bands 2602, 2604, and/or 2606 in the user interface 2600. As depicted, the graphical element 2630 includes the following detailed information: “names: Derriengue; week: 33; date: 16 Aug. 2015 06:00; event: Derriengue; event_type: Condition”.

In some embodiments, the user may select one of the squares representing an event. The computing device may transmit the selection to a server. The computing device may receive an action instruction that is generated using the taxonomy of ontological medical knowledge based on a patient graph for the person associated with the event. The computing device may present the action instruction.

IN FIG. 26B, the user may select another one of the squares representing a second event in the clustered event episode bundles to view detailed information about that second event. As depicted, the user selected a second square 2640 for a second event in a different row of the band 2502. The selection may cause a graphical element 2630 to be presented on the user interface 2600 and the graphical element 2630 may overlay or occlude a portion of one or more of the bands 2602, 2604, and/or 2606 in the user interface 2600. As depicted, the graphical element 2630 includes the following detailed information for the second event: “names: 10 ML rabies immu . . . ; week: 37; date: 8 Sep. 2015 04:00; event: 10 ML rabies immune globulin, human 150 UNT/ML Injection; event_type: Medication”.

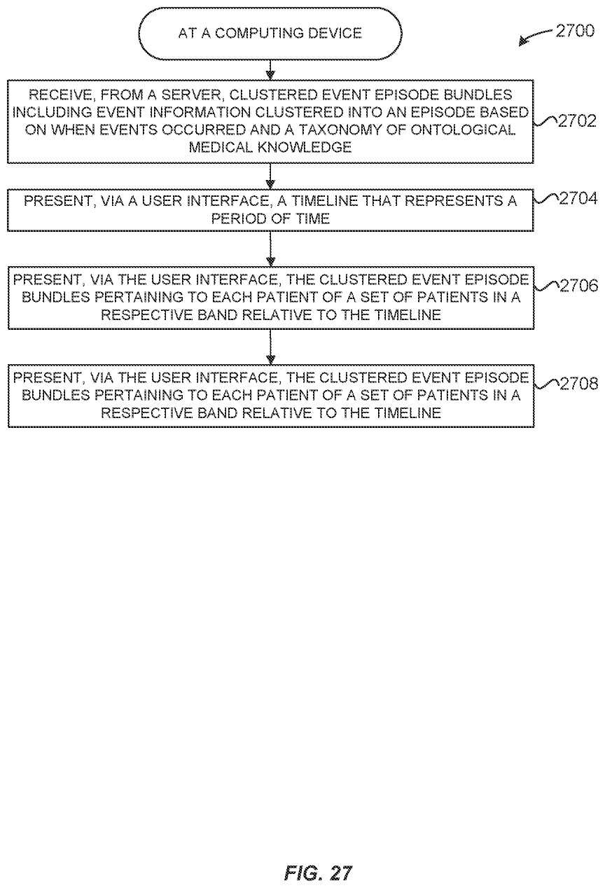

FIG. 27 shows an example method 2700 for presenting clustered event episode bundles on a timeline, in accordance with various embodiments. In some embodiments, the method 2700 is implemented on a computing device. In some embodiments, the computing device is the user device 104 and/or a computing device operated by the service provider 112 and/or the facility 114. In some embodiments, the computing device that performs the method 2700 may include one or more of the computing devices 2900 of FIG. 29. The method 2700 may include operations that are implemented in computer instructions stored in a memory and executed by a processor of a computing device.

At block 2702, the processing device may receive, from a server (e.g., the cognitive intelligence platform 102), clustered event episode bundles including event information clustered into an episode based on when events occurred and a taxonomy of ontological medical knowledge. In some embodiments, the types of events include medical conditions, medications, observations, or some combination thereof.

At block 2704, the processing device may present, via a user interface, a timeline that represents a period of time.

At block 2706, the processing device may present, via the user interface, the clustered event episode bundles pertaining to each patient of a set of patients in a respective band relative to the timeline. In some embodiments, the processing device may present different types of the events as objects in different respective rows of each band, as depicted in FIGS. 26A and 26B. In some embodiments, the processing device may provide an option to interact with the objects to obtain detailed information within the user interface. In some embodiments, the processing device may present an indication of a duration of each clustered event episode bundle presented in each respective band and an indication of an amount of time elapsed between clustered event episode bundles in each respective band.

At block 2708, the processing device may present, via the user interface, the clustered event episode bundles pertaining to each patient of a set of patients in a respective band relative to the timeline.

In some embodiments, the processing device may receive, from an input device (e.g., peripheral device) of a computing device of a medical personnel, an input pertaining to an event in a clustered event episode bundle for a patient, as depicted in FIG. 26A. The processing device may present detailed information pertaining to the event on the user interface, as depicted in FIG. 26A. The detailed information may include a name of the event, an indication of when the event occurred, a description of the event, a type of the event, or some combination thereof. In some embodiments, the detailed information may be included in a graphical element that overlays or occludes a portion of the user interface presenting the band in which the event is included.

Such a technique may improve the user experience using a computing device by providing an enhanced user interface that enables seeing detailed information for multiple events on one user interface without having to search for the various events on other user interfaces. To that end, the techniques employed by the clustered episode event bundles reduces network resources and/or processing resources because the information for each event are clustered and provided for a bundle for each person in a respective band and the user does not have to search for multiple events individually, thereby reducing the number of searches performed to obtain the desired medical history information.

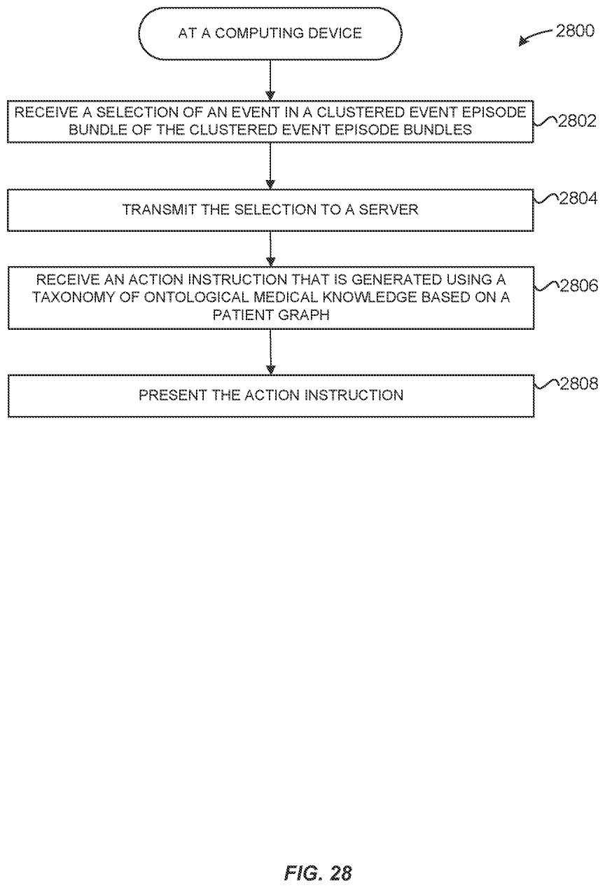

FIG. 28 shows an example method 2800 for receiving a selection of an event in a clustered event episode bundle and receiving an action instruction that is generated based on the event, in accordance with various embodiments. In some embodiments, the method 2800 is implemented on a computing device. In some embodiments, the computing device is the user device 104 and/or a computing device operated by the service provider 112 and/or the facility 114. In some embodiments, the computing device that performs the method 2800 may include one or more of the computing devices 2900 of FIG. 29. The method 2800 may include operations that are implemented in computer instructions stored in a memory and executed by a processor of a computing device.

At block 2802, the processing device may receive a selection of an event in a clustered event episode bundle. The clustered event episode bundle may be included in a band representing a medical condition of a person. The selection may be received via the user interface presented on the computing device of the medical personnel depicted in FIG. 26A or 26B. At block 2804, the processing device may transmit the selection to a server (e.g., included in the cognitive intelligence platform 102).

At block 2806, the processing device may receive an action instruction that is generated using a taxonomy of ontological medical knowledge based on a patient graph. The patient graph for the person may represent the various interactions and/or encounters with the medical condition. Each of the clustered episode events included in the band for the bundle may be represented in the patient graph. The patient graph may represent a subset of health artifacts and relationships between the subset of health artifacts for the medical condition. The taxonomy of ontological medical knowledge may represent a superset of health artifacts and relationships between the superset of health artifacts for the medical condition. The comparison between the patient graph and the taxonomy of ontological medical knowledge may be performed by overlaying the patient graph on the taxonomy of ontological medical knowledge (e.g., knowledge graph). The processing device may determine that the patient graph is lacking a particular health artifact in view of the knowledge graph. For example, the person has not had a particular test for the medical condition or been prescribed a particular medication for the medical condition. Accordingly, the processing device may generate an action instruction pertaining to the gap in diagnosis or treatment.

At block 2808, the processing device may present the action instruction on the computing device of the user (e.g., medical personnel). The action instruction may recommend prescribing the person a certain medication, scheduling an appointment, referring the person to another specialist, performing a certain test, performing a certain procedure, or the like.

FIG. 29 illustrates a detailed view of a computer system 2900, which can perform any one or more of the methods described herein. In particular, the detailed view illustrates various components that can be included in the user device 104 illustrated in FIG. 1, as well as the several computing devices implementing the cognitive intelligence platform 102. Further, the computer system 2900 may include components that can be included in computing devices operated by the service provider 112 and/or the facility 114. The computer system 2900 may correspond to any component in the cognitive intelligence platform 102 (e.g., knowledge cloud 106, cognitive agent 110, critical thinking engine 108, natural language database 122, etc.).

The computer system 2900 may be connected (e.g., networked) to other computer systems in a LAN, an intranet, an extranet, or the Internet. The computer system 2900 may operate in the capacity of a server in a client-server network environment. The computer system 2900 may be a server, a personal computer (PC), a tablet computer, a wearable (e.g., wristband), a set-top box (STB), a personal Digital Assistant (PDA), a mobile phone, a camera, a video camera, or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 2900 includes a processing device 2902, a main memory device 2904 (e.g., read-only memory (ROM), solid state drive (SSD), flash memory, dynamic random access memory (DRAM) such as synchronous DRAM (SDRAM)), a static memory 2906 (e.g., solid state drive (SSD), flash memory, static random access memory (SRAM)), and a data storage device 2908, which communicate with each other via a bus 2910.

Processing device 2902 represents one or more general-purpose processing devices such as a microprocessor, central processing unit, or the like. More particularly, the processing device 2902 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 2902 may also be one or more special-purpose processing devices such as an application specific integrated circuit (ASIC), a field programmable gate array (FPGA), a digital signal processor (DSP), network processor, or the like. The processing device 2902 is configured to execute instructions for performing any of the operations and steps discussed herein.

The computer system 2900 may further include a network interface 2912. The computer system 2900 also may include a video display 2914 (e.g., a liquid crystal display (LCD) or a cathode ray tube (CRT)), one or more input devices 2916 (e.g., a keyboard, touchscreen, and/or a mouse), and one or more speakers 2918 (e.g., a speaker). In one illustrative example, the video display 2914 and the input device(s) 2916 may be combined into a single component or device (e.g., an LCD touch screen).

The data storage device 2916 may include a computer-readable medium 2920 on which the instructions 2922 embodying any one or more of the methodologies or functions described herein are stored. The instructions 2922 may also reside, completely or at least partially, within the main memory device 2904 and/or within the processing device 2902 during execution thereof by the computer system 2900. As such, the main memory device 2904 and the processing device 2902 also constitute computer-readable media. The instructions 2922 may further be transmitted or received over a network via the network interface 2912.

While the computer-readable storage medium 2920 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The various aspects, embodiments, implementations or features of the described embodiments can be used separately or in any combination. Various aspects of the described embodiments can be implemented by software, hardware or a combination of hardware and software. The described embodiments can also be embodied as computer readable code on a computer readable medium. The computer readable medium is any data storage device that can store data which can thereafter be read by a computer system. Examples of the computer readable medium include read-only memory, random-access memory, CD-ROMs, DVDs, magnetic tape, hard disk drives, solid-state drives, and optical data storage devices. The computer readable medium can also be distributed over network-coupled computer systems so that the computer readable code is stored and executed in a distributed fashion.

Consistent with the above disclosure, the examples of systems and method enumerated in the following clauses are specifically contemplated and are intended as a non-limiting set of examples.

Clause 1. A method for operating an artificial intelligence engine, the method comprising:

determining a shared medical condition state for a subset of a population of people;

segmenting the population of the people into a registry of people based on the shared medical condition state;

determining an action to perform for the registry of the people based on comparing the shared medical condition state to ontological medical information; and

autonomously performing, by the artificial intelligence engine, the action for the registry of the people.

Clause 2. The method of any preceding clause, wherein the action includes scheduling an appointment for a person in the registry of the people, sending an alert to a computing device of the person, calling a computing device of the person, causing information to be presented via an audio device of the computing device of the person, or some combination thereof.

Clause 3. The method of any preceding clause, wherein the shared medical condition state comprises a type of medical condition, a severity of the medical condition, a stage of the medical condition, encounters previously engaged by the people to treat the medical condition, age of the people, a prevalence of the medical condition, a number of the encounters, or some combination thereof.

Clause 4. The method of any preceding clause, further comprising causing the shared medical condition state, in conjunction with a recommendation pertaining to the action, to be presented on a computing device of a medical personnel.

Clause 5. The method of any preceding clause, further comprising causing information pertaining to the registry of people to be presented on a computing device of a medical personnel.

Clause 6. The method of any preceding clause, wherein the ontological medical information is included in a knowledge graph that comprises health artifacts for a medical condition and relationships between the health artifacts.

Clause 7. The method of any preceding clause, further comprising:

segmenting the population of the people into a second registry of the people based on a second shared medical condition state;

determining a second action to perform based on comparing the second shared medical condition state to the ontological medical information; and

performing the second action, wherein the second action is different than the action.

Clause 8. The method of any preceding clause, further comprising:

tracking encounters with a medical condition associated with the shared medical condition state for each person of the population of the people.

Clause 9. The method of any preceding clause, further comprising:

determining a second action to perform for a person in the registry of the people based on comparing the encounters to the ontological medical information; and

performing the second action for the person.

Clause 10. The method of any preceding clause, further comprising:

determining a second action to perform for a person in the registry of the people based on comparing the encounters to the ontological medical information; and

performing the second action for the person.

Clause 11. A system comprising:

a memory device storing instructions that implement an artificial intelligence engine; and

a processing device communicatively coupled to the memory device, the processing device to execute the instructions to cause the artificial intelligence engine to:
determine a shared medical condition state for a subset of a population of people;
segment the population of the people into a registry of people based on the shared medical condition state;
determine an action to perform for the registry of the people based on comparing the shared medical condition state to ontological medical information; and

autonomously perform the action for the registry of the people.

Clause 12. The system of any preceding clause, wherein the action includes scheduling an appointment for a person in the registry of the people, sending an alert to a computing device of the person, calling a computing device of the person, causing information to be presented via an audio device of the computing device of the person, or some combination thereof.

Clause 13. The system of any preceding clause, wherein the shared medical condition state comprises a type of medical condition, a severity of the medical condition, a stage of the medical condition, encounters previously engaged by the people to treat the medical condition, age of the people, a prevalence of the medical condition, a number of the encounters, or some combination thereof.

Clause 14. The system of any preceding clause, wherein the processing device is further to cause the shared medical condition state, in conjunction with a recommendation pertaining to the action, to be presented on a computing device of a medical personnel.

Clause 15. The system of any preceding clause, wherein the processing device is further to cause information pertaining to the registry of people to be presented on a computing device of a medical personnel.

Clause 16. The system of any preceding clause, wherein the ontological medical information is included in a knowledge graph that comprises health artifacts for a medical condition and relationships between the health artifacts.

Clause 17. The system of any preceding clause, wherein the processing device is further to:

segment the population of the people into a second registry of the people based on a second shared medical condition state;

determine a second action to perform based on comparing the second shared medical condition state to the ontological medical information; and

perform the second action, wherein the second action is different than the action.

Clause 18. The system of any preceding clause, wherein the processing device is further to:

track encounters with a medical condition associated with the shared medical condition state for each person of the population of the people.

Clause 19. The system of any preceding clause, wherein the processing device is further to:

determine a second action to perform for a person in the registry of the people based on comparing the encounters to the ontological medical information; and

perform the second action for the person.

Clause 20. A tangible, non-transitory computer-readable medium storing instructions that, when executed, cause a processing device to:

determine a shared medical condition state for a subset of a population of people;

segment the population of the people into a registry of people based on the shared medical condition state;

determine an action to perform for the registry of the people based on comparing the shared medical condition state to ontological medical information; and

autonomously perform the action for the registry of the people.

Clause 21. The tangible, non-transitory computer-readable medium of any preceding claim, wherein the action includes scheduling an appointment for a person in the registry of the people, sending an alert to a computing device of the person, calling a computing device of the person, causing information to be presented via an audio device of the computing device of the person, or some combination thereof.

Clause 22. A method for presenting a user interface including clustered event episode bundles pertaining to a medical condition, the method comprising:

receiving, from a server, the clustered event episode bundles comprising event information clustered into an episode based on when events occurred and a taxonomy of ontological medical knowledge;

presenting, via the user interface, a timeline that represents a period of time; and

presenting, via the user interface, the clustered event episode bundles pertaining to each patient of a plurality of patients in a respective band relative to the timeline.

Clause 23. The method of any preceding claim, further comprising:

receiving, from a peripheral device of a computing device, an input pertaining to an event in a clustered event episode bundle for a patient;

presenting detailed information pertaining to the event on the user interface.

Clause 24. The method of any preceding claim, wherein the detailed information comprises a name of the event, an indication of when the event occurred, a description of the event, a type of the event, or some combination thereof.

Clause 25. The method of any preceding claim, further comprising including the detailed information in a graphical element that occludes a portion of the user interface presenting the band in which the event is included.

Clause 26. The method of any preceding claim, wherein types of events comprise conditions, medications, observations, or some combination thereof.

Clause 27. The method of any preceding claim, further comprising:

receiving a selection of an event in a clustered event episode bundle of the clustered event episode bundles;

transmitting the selection to the server;

receiving an action instruction that is generated using the taxonomy of ontological medical knowledge based on a patient graph; and

presenting the action instruction.

Clause 28. The method of any preceding claim, further comprising presenting different types of the events as objects in different respective rows of each band.

Clause 29. The method of any preceding claim, further comprising providing an option to interact with the objects to obtain detailed information within the user interface.

Clause 30. The method of any preceding claim, further comprising presenting an indication of a duration of each clustered event episode bundle presented in each respective band and an indication of an amount of time elapsed between clustered event episode bundles in each respective band.

Clause 31. A system for presenting a user interface including clustered event episode bundles pertaining to a medical condition, the system comprising:

a memory device storing instructions; and

a processing device communicatively coupled to the memory device, the processing device executes the instructions to:
receive, from a server, the clustered event episode bundles comprising event information clustered into an episode based on when events occurred and a taxonomy of ontological medical knowledge;
present, via the user interface, a timeline that represents a period of time; and
present, via the user interface, the clustered event episode bundles pertaining to each patient of a plurality of patients in a respective band relative to the timeline.

Clause 32. The system of any preceding claim, wherein the processing device is further to:

receive, from a peripheral device of a computing device, an input pertaining to an event in a clustered event episode bundle for a patient;

present detailed information pertaining to the event on the user interface.

Clause 33. The system of any preceding claim, wherein the detailed information comprises a name of the event, an indication of when the event occurred, a description of the event, a type of the event, or some combination thereof.

Clause 34. The system of any preceding claim, wherein the processing device is further to include the detailed information in a graphical element that occludes a portion of the user interface presenting the band in which the event is included.

Clause 35. The system of any preceding claim, wherein types of events comprise conditions, medications, observations, or some combination thereof.

Clause 36. The system of any preceding claim, wherein the processing device is further to:

receive a selection of an event in a clustered event episode bundle of the clustered event episode bundles;

transmit the selection to the server;

receive an action instruction that is generated using the taxonomy of ontological medical knowledge based on a patient graph; and

present the action instruction.

Clause 37. The system of any preceding claim, wherein the processing device is further to present different types of the events as objects in different respective rows of each band.

Clause 38. The system of any preceding claim, wherein the processing device is further to provide an option to interact with the objects to obtain detailed information within the user interface.

Clause 39. The system of any preceding claim, wherein the objects comprise a square, a rectangle, a circle, an oval, a diamond, a triangle, a hexagon, an octagon, or some combination thereof.

Clause 40. A tangible, non-transitory computer-readable medium including instructions that, when executed, cause a processing device to:

receive, from a server, the clustered event episode bundles comprising event information clustered into an episode based on when events occurred and a taxonomy of ontological medical knowledge;

present, via a user interface, a timeline that represents a period of time; and

present, via the user interface, the clustered event episode bundles pertaining to each patient of a plurality of patients in a respective band relative to the timeline.

Clause 41. A tangible, non-transitory computer-readable medium of any preceding claim: wherein the processing device is further to:

receive, from a peripheral device of a computing device, an input pertaining to an event in a clustered event episode bundle for a patient;

present detailed information pertaining to the event on the user interface.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A method for presenting a user interface including clustered event episode bundles pertaining to a medical condition, the method comprising:
receiving, from a server, the clustered event episode bundles comprising event information clustered into an episode based on when events occurred and a taxonomy of ontological medical knowledge;
presenting, via the user interface, a timeline that represents a period of time; and
presenting, via the user interface, the clustered event episode bundles pertaining to each patient of a plurality of patients in a respective band relative to the timeline.

2. The method of claim 1, further comprising:
receiving, from a peripheral device of a computing device, an input pertaining to an event in a clustered event episode bundle for a patient;
presenting detailed information pertaining to the event on the user interface.

3. The method of claim 2, wherein the detailed information comprises a name of the event, an indication of when the event occurred, a description of the event, a type of the event, or some combination thereof.

4. The method of claim 2, further comprising including the detailed information in a graphical element that occludes a portion of the user interface presenting the band in which the event is included.

5. The method of claim 1, wherein types of events comprise conditions, medications, observations, or some combination thereof.

6. The method of claim 1, further comprising:
receiving a selection of an event in a clustered event episode bundle of the clustered event episode bundles;
transmitting the selection to the server;
receiving an action instruction that is generated using the taxonomy of ontological medical knowledge based on a patient graph; and
presenting the action instruction.

7. The method of claim 1, further comprising presenting different types of the events as objects in different respective rows of each band.

8. The method of claim 7, further comprising providing an option to interact with the objects to obtain detailed information within the user interface.

9. The method of claim 7, further comprising presenting an indication of a duration of each clustered event episode bundle presented in each respective band and an indication of an amount of time elapsed between clustered event episode bundles in each respective band.

10. A system for presenting a user interface including clustered event episode bundles pertaining to a medical condition, the system comprising:
a memory device storing instructions; and
a processing device communicatively coupled to the memory device, the processing device executes the instructions to:
receive, from a server, the clustered event episode bundles comprising event information clustered into an episode based on when events occurred and a taxonomy of ontological medical knowledge;
present, via the user interface, a timeline that represents a period of time; and
present, via the user interface, the clustered event episode bundles pertaining to each patient of a plurality of patients in a respective band relative to the timeline.

11. The system of claim 10, wherein the processing device is further to:
receive, from a peripheral device of a computing device, an input pertaining to an event in a clustered event episode bundle for a patient;
present detailed information pertaining to the event on the user interface.

12. The system of claim 11, wherein the detailed information comprises a name of the event, an indication of when the event occurred, a description of the event, a type of the event, or some combination thereof.

13. The system of claim 11, wherein the processing device is further to include the detailed information in a graphical element that occludes a portion of the user interface presenting the band in which the event is included.

14. The system of claim 10, wherein types of events comprise conditions, medications, observations, or some combination thereof.

15. The system of claim 10, wherein the processing device is further to:
receive a selection of an event in a clustered event episode bundle of the clustered event episode bundles;
transmit the selection to the server;
receive an action instruction that is generated using the taxonomy of ontological medical knowledge based on a patient graph; and
present the action instruction.

16. The system of claim 10, wherein the processing device is further to present different types of the events as objects in different respective rows of each band.

17. The system of claim 16, wherein the processing device is further to provide an option to interact with the objects to obtain detailed information within the user interface.

18. The system of claim 16, wherein the objects comprise a square, a rectangle, a circle, an oval, a diamond, a triangle, a hexagon, an octagon, or some combination thereof.

19. A tangible, non-transitory computer-readable medium including instructions that, when executed, cause a processing device to:
receive, from a server, the clustered event episode bundles comprising event information clustered into an episode based on when events occurred and a taxonomy of ontological medical knowledge;
present, via a user interface, a timeline that represents a period of time; and
present, via the user interface, the clustered event episode bundles pertaining to each patient of a plurality of patients in a respective band relative to the timeline.

20. The computer-readable medium of claim 19, wherein the processing device is further to:
receive, from a peripheral device of a computing device, an input pertaining to an event in a clustered event episode bundle for a patient;
present detailed information pertaining to the event on the user interface.
